(19) United States
(12) Patent Application Publication
(10) Pub. No.: US 2022/0002307 A1
(43) Pub. Date: Jan. 6, 2022

(54) FGFR4 INHIBITOR AND USE THEREOF

(71) Applicant: BETTA PHARMACEUTICALS CO., LTD, Zhejiang (CN)

(72) Inventors: Jinheng GAO, Beijing (CN); Zhongxin SUN, Beijing (CN); Yun ZHANG, Beijing (CN); Xiaofeng XU, Beijing (CN); Xiangyong LIU, Beijing (CN); Jiabing WANG, Beijing (CN); Lieming DING, Zhejiang (CN)

(21) Appl. No.: 17/280,309

(22) PCT Filed: Sep. 26, 2019
(86) PCT No.: PCT/CN2019/108298

§ 371 Date: Mar. 26, 2021

(30) Foreign Application Priority Data
Sep. 27, 2018 (CN) PCT/CN2018/107902

(51) Int. Cl. C07D 487/14; C07D 471/04; C07D 471/14; C07D 491/147; C07D 491/22; C07D 495/14; C07D 513/14; A61P 35/00

(52) U.S. Cl. CPC C07D 487/14; C07D 471/04; C07D 471/14; A61P 35/00; C07D 491/22; C07D 495/14; C07D 513/14; C07D 491/147

(57) ABSTRACT

Disclosed is a compound as a fibroblast growth factor receptor 4 (FGFR4) inhibitor (as shown in formula (I)), and a pharmaceutical composition thereof and a preparation method therefor, as well as the use of same in the treatment of FGFR4-mediated diseases. The above-mentioned compounds act by participating in a number of processes, such as regulating cell proliferation, apoptosis, migration, neovascularization.

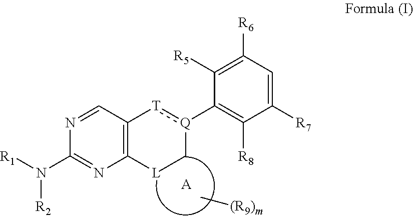

### FIELD OF THE INVENTION

The present invention relates to a series of compound which act as inhibitors of fibroblast growth factor receptors 4 (FGFR4), as well as the preparation method and pharmaceutical composition thereof. The present invention further relates to a use of the above compounds or pharmaceutical composition thereof in the treatment of FGFR4-mediated disorders.

### BACKGROUND OF THE INVENTION

Protein kinases are enzymes that catalyze the phosphorylation of proteins, in most instances, this phosphorylation occurs on the residues of the serine (ser), threonine (thr) and tyrosine (tyr) of the protein. Many aspects of cell life processes (eg. cell growth, differentiation, proliferation, cell cycle and survival) are dependent on the activity of protein kinase. Furthermore, many diseases (eg. cancer and inflammation) are associated with the abnormal activity of protein kinase.

It has been found that Protein Tyrosine Kinase, PTK have more than 100 family members so far, which play an important role in regulating cell differentiation, growth and proliferation. According to the structure of the PTK, it can be divided into two types: receptor PTK which is also called as transmembrane PTK and non-receptor PTK which is also called as intracellular PTK

Fibroblast growth factor receptors (FGFR) is a member of receptor tyrosine kinase (RTK) superfamily, which regulates the cell proliferation, differentiation and migration in different tissues through the complex signal transmission pathways by combining with fibroblast growth factor (FGF) (Jouanneau J et al. Oncogene, 1999, 18:327-333). FGFR is a single-chain glycoprotein consisted of an extracellular region, a single transmembrane region and a tyrosine kinase region in the cytoplasm. The extracellular region is composed of a leader peptide and three immunoglobulin domains. The FGFRs family comprises FGFR1, FGFR2, FGFR3 and FGFR4. The FGFR4 gene is located at 5q35.1 of chromosome, which is about 11.3 kb in length with 18 exons (Kostrzewa M. Mammalian genome, 1998, 9(2): 131-135). FGFR4 protein is an important member of the FGFR tyrosine kinase family, its' 388th amino acid is located in the highly conserved transmembrane region of RTK structure, the changes in the pathophysiological function of FGFR4 protein caused by the changes of this structural can enhance the activity of tyrosine kinase. FGFR4 protein is a type of transmembrane tyrosine kinase receptor with autophosphorylation activity, which plays an important role in embryonic development, tissue repair and angiogenesis. (Eswarakumar V P et al. Cytokine Growth Factor Rev, 2005, 16(2): 139-149).

FGFR4 signaling pathway: Mediated by heparin or heparinoid, the ligand binds to FGFR4, causing the FGFR4 monomer to dimerize, with the tyrosine phosphorylation of the C-terminus of the cytoplasm, the kinase insertion region and the proximal membrane region, FGFR4 is activated by the phosphorylation of the kinase domain of A loop (activation loop). (Schlessinger J et al. Mol Cell, 2000, 6:743-750). The activated FGFR4 mainly has two intracellular agents which are Phospholipase C and FGF receptor substrate 2 (FRS2) (Dailey L et al. Cytokine Growth Factor Rev, 2005, 16:233-247).

When FGFR4 is activated, the Src homology region 2 (SH2) domain of phospholipase C binds to its activated C-terminal tyrosine, causing the phosphorylation of the PLC and binds to the C-terminal tyrosine site. The activated PLC hydrolyzes its substrate 4,5-diphosphate phosphatidylinositol (PIP2) to form diacylglycerol (DAG) and inositol triphosphate (IP3). IP3 binds to specific receptors in the cell to stimulate the intracellular calcium pool to release Ca²⁺, Ca²⁺ binds to calmodulin to activate Ca²⁺/calmodulin-dependent protein kinase. Besides, both Ca²⁺ and diacylglycerol can activate members of the protein kinase C family. In addition to activating transcription factors, the secondary signal generated by PIP2 hydrolysis can also activate a variety of intracellular reactions.

SOS protein binds to the Src homology region 3 (SH3) domain of growth factor receptor bound 2 (Grb2) to form a Grb2/SOS complex, which can binds to FGFR4 or FGFR substrate 2α (FRS2α), wherein FRS2α is connected with phosphotyrosine binding domain (PTB), promoting the exchange of guanosine on Ras to make Ras become Ras-GTP to starting the downstream MAPK signaling pathway.

The autophosphorylation of FGFR4 activates JAK family factors (JAK), the activated JAK cause the phosphorylation of specific signal protein adsorption site on FGFR4, this site can be the docking sites of signal transducer and activator of transcription (STAT) and other signaling molecules. The C-terminal tyrosine residue of the STAT protein is phosphorylated by JAK when STAT protein is absorbed by FGFR4 docking site, and the phosphorylated STAT protein separates from the receptor to form a stable homodimer or heterodimer and then is transferred to the nucleus to interact with gamma interferon activation site (GAS) enhancer family members to activate the transcription of target genes.

Small molecules FGFR4 inhibitors inhibit the proliferation signal mediated by FGFR4 by blocking the combination of the extracellular ligand molecules with receptors or the transmission of intracellular kinase signals. There are many types of FGFR4 inhibitors currently under development, the FGFR4 selective inhibitor AZ709 developed by AstraZeneca shows a good inhibitory effect on cells expressing FGF19 or FGFR4 at high level in vitro experiments, but there's no obvious effect in the in vivo experiments. The FGFR4 selective inhibitor FGF401 developed by Novartis can target FGFR4 specifically to treat the malignant tumors such as liver cancer caused by the overexpression of the FGFR4. The FGFR4 specific inhibitor H3B6527 developed by H3 Biomedicine has a strong anti-tumor activity on FGF19 gene-amplified cells, and isn't found any bile acid-related adverse reactions in the mouse and monkey animal models. Blueprint Medicine has developed and reported a FGFR4-specific inhibitor BLU554 to treat liver cancer and cholangiocarcinoma with overexpression of FGFR4.

With the in-depth study of the structure and the function of FGFR4 and the interaction with other genes, the FGFR4 inhibitors with good specificity and therapeutic effect and low adverse reactions will be developed, and the use of FGFR4 molecular targeted therapy for tumors will be very meaningful.

### SUMMARY OF THE INVENTION

The present invention relates to compounds as an inhibitor of fibroblast growth factor receptors 4 (FGFR4), or pharmaceutical acceptable salts, solvates, chelates, non-covalent complexes or prodrugs thereof. The compounds of the present invention have a general structure as Formula (I).

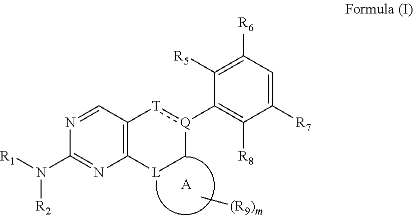

wherein,

[symbol] is a single bond or a double bond;

L, Q or T is each independently selected from the group consisting of O, N, C, CH, CH₂ and CR₁₇;

ring A is C₆₋₁₀aryl, substituted C₆₋₁₀aryl, C₅₋₁₀heteroaryl, substituted C₅₋₁₀heteroaryl, C₅₋₁₀heterocyclyl, substituted C₅₋₁₀heterocyclyl, wherein the C₅₋₁₀heteroaryl or C₅₋₁₀heterocyclyl optionally containing 1, 2 or 3 heteroatoms independently selected from N, O and S;

R₁ is selected from the group consisting of hydrogen, halogen, C₁₋₈alkyl, substituted C₁₋₈alkyl, C₁₋₈alkoxy, substituted C₁₋₈alkoxy, C₂₋₈alkenyl, substituted C₂₋₈alkenyl, C₂₋₈alkynyl and substituted C₂₋₈alkynyl;

R₂ is selected from the group consisting of C₃₋₁₀cycloalkyl, substituted C₃₋₁₀cycloalkyl, C₃₋₁₀heterocyclyl, substituted C₃₋₁₀heterocyclyl, C₆₋₁₀aryl, substituted C₆₋₁₀aryl, C₆₋₁₀heteroaryl and substituted C₆₋₁₀heteroaryl, wherein the C₃₋₁₀heterocyclyl or C₆₋₁₀heteroaryl optionally containing 1 or 2 heteroatoms selected from N and O;

R₂ is optionally substituted with 1-2 R₁₃ substituents;

R₁₃ is selected from the group consisting of hydroxyl, halogen, C₁₋₈alkyl, substituted C₁₋₈alkyl, C₂₋₈alkenyl, substituted C₂₋₈alkenyl, C₂₋₈alkynyl, substituted C₂₋₈alkynyl, C₁₋₈alkoxy, substituted C₁₋₈alkoxy, C₆₋₁₀aryl, substituted C₆₋₁₀aryl, C₃₋₁₀cycloalkyl, substituted C₃₋₁₀cycloalkyl, C₃₋₁₀heterocyclyl, substituted C₃₋₁₀heterocyclyl, C₅₋₁₀heteroaryl, substituted C₅₋₁₀heteroaryl, —NR₁₄R₁₅ and —CO—R₁₆, wherein C₃₋₁₀heterocyclyl or C₅₋₁₀heteroaryl optionally containing 1, 2 or 3 heteroatoms selected from N and O;

R₁₃ is optionally substituted with 0-1 R₁₈ substituent;

R₁₈ is selected from the group consisting of hydroxyl, halogen, C₁₋₈alkyl, substituted C₁₋₈alkyl, C₂₋₈alkenyl, substituted C₂₋₈alkenyl, C₂₋₈alkynyl, substituted C₂₋₈alkynyl, C₁₋₈alkoxy, substituted C₁₋₈alkoxy, C₆₋₁₀aryl, substituted C₆₋₁₀aryl, C₃₋₁₀cycloalkyl, substituted C₃₋₁₀cycloalkyl, C₃₋₁₀heterocyclyl, substituted C₃₋₁₀heterocyclyl, C₅₋₁₀heteroaryl and substituted C₅₋₁₀heteroaryl, wherein C₃₋₁₀heterocyclyl or C₅₋₁₀heteroaryl containing 1, 2 or 3 heteroatoms selected from N and O;

R₅, R₆, R₇ and R₈ is independently selected from the group consisting of hydroxyl, halogen, C₁₋₈alkoxy, substituted C₁₋₈alkoxy, C₁₋₈alkyl, substituted C₁₋₈alkyl, C₂₋₈alkenyl, substituted C₂₋₈alkenyl, C₂₋₈alkynyl, substituted C₂₋₈alkynyl, C₃₋₈cycloalkyl, substituted C₃₋₈cycloalkyl, C₆₋₁₀aryl, substituted C₆₋₁₀aryl, C₅₋₁₀heteroaryl, substituted C₅₋₁₀heteroaryl, C₃₋₁₀heterocyclyl and substituted C₃₋₁₀ heterocyclyl;

R₉ is selected from the group consisting of H, halogen, amino, cyano, C₁₋₈alkyl, substituted C₁₋₈alkyl, C₁₋₈alkoxy, substituted C₁₋₈alkoxy, C₂₋₈alkenyl, substituted C₂₋₈alkenyl, C₂₋₈alkynyl, substituted C₂₋₈alkynyl, C₆₋₁₀aryl, substituted C₆₋₁₀aryl, C₃₋₈cycloalkyl, substituted C₃₋₈cycloalkyl, C₃₋₁₀heterocyclyl, substituted C₃₋₁₀heterocyclyl, C₅₋₁₀heteroaryl and substituted C₅₋₁₀heteroaryl;

R₉ is optionally substituted with 0-1 R₁₀ substituent;

R₁₀ is selected from the group consisting of hydroxyl, halogen, C₁₋₈alkyl, substituted C₁₋₈alkyl, C₂₋₈alkenyl, substituted C₂₋₈alkenyl, C₂₋₈alkynyl, substituted C₂₋₈alkynyl, C₁₋₈alkoxy, substituted C₁₋₈alkoxy, C₆₋₁₀aryl, substituted C₆₋₁₀aryl, C₃₋₈cycloalkyl, substituted C₃₋₈cycloalkyl, C₃₋₁₀heterocyclyl, substituted C₃₋₁₀heterocyclyl, C₅₋₁₀heteroaryl, substituted C₅₋₁₀heteroaryl, —CO—R₁₆ and —(CH₂)ₙNR₁₁R₁₂;

R₁₁ or R₁₂ is optionally selected from the group consisting of H, C₁₋₈alkyl, substituted C₁₋₈alkyl, C₂₋₈alkenyl, substituted C₂₋₈alkenyl, C₂₋₈alkynyl, substituted C₂₋₈alkynyl, C₁₋₈alkoxy, substituted C₁₋₈alkoxy, C₃₋₈cycloalkyl, substituted C₃₋₈cycloalkyl, C₆₋₁₀aryl, substituted C₆₋₁₀aryl, C₅₋₁₀heteroaryl, substituted C₅₋₁₀heteroaryl, C₃₋₁₀heterocyclyl and substituted C₃₋₁₀ heterocyclyl;

R₁₄ or R₁₅ is optionally selected from the group consisting of H, C₁₋₈alkyl, substituted C₁₋₈alkyl, C₂₋₈alkenyl, substituted C₂₋₈alkenyl, —CO—C₂₋₈alkenyl, substituted —CO—C₂₋₈alkenyl, C₂₋₈alkynyl, substituted C₂₋₈alkynyl, C₃₋₁₀cycloalkyl, substituted C₃₋₁₀cycloalkyl, C₆₋₁₀aryl, substituted C₆₋₁₀aryl, C₅₋₁₀heteroaryl, substituted C₅₋₁₀ heteroaryl, C₃₋₁₀heterocyclyl and substituted C₃₋₁₀heterocyclyl;

R₁₆ is optionally selected from the group consisting of C₁₋₈alkyl, substituted C₁₋₈ alkyl, C₂₋₈alkenyl, substituted C₂₋₈alkenyl, C₂₋₈alkynyl, substituted C₂₋₈alkynyl, C₃₋₁₀cycloalkyl, substituted C₃₋₁₀cycloalkyl, C₆₋₁₀aryl, substituted C₆₋₁₀aryl, C₅₋₁₀heteroaryl, substituted C₅₋₁₀heteroaryl, C₃₋₁₀heterocyclyl, substituted C₃₋₁₀heterocyclyl and —NR₁₁R₁₂;

R₁₇ is selected from the group consisting of oxo, C₁₋₈alkyl, substituted C₁₋₈alkyl, C₂₋₈alkenyl, substituted C₂₋₈alkenyl, C₂₋₈alkynyl, substituted C₂₋₈alkynyl, C₃₋₁₀cycloalkyl, substituted C₃₋₁₀cycloalkyl, C₆₋₁₀aryl, substituted C₆₋₁₀aryl, C₅₋₁₀ heteroaryl, substituted C₅₋₁₀ heteroaryl, C₃₋₁₀ heterocyclyl, substituted C₃₋₁₀ heterocyclyl, or R₁₇ is C₃₋₁₀ cycloalkyl, taken together with the carbon atom to which they are attached form a spiro ring;

M is 0 or 1;

N is 0, 1 or 2;

and provided that:

if ring A is a 5-member heteroaryl containing 2 or 3 N atoms, then R₂ is vinylamide substituted 6-member heterocyclyl comprising oxygen heteroatom, wherein vinylamide substituted 6-member heterocyclyl optionally substituted with 0-1 R₁₃ substituent;

if T is CR₁₇, and R₁₇ is oxo, then ring A is not a 5-member heteroaryl comprising N heteroatom.

The present invention further provides some preferred technical solutions with regard to the compound of formula (I):

In some embodiments, the L, Q and T of formula (I) are selected from the following groups:
(i) L is C, Q is N, T is CH₂;
(ii) L is C, Q is N, T is C;
(iii) L is C, Q is C, T is N;
(iv) L is C, Q is N, T is CH;
(v) L is N, Q is N, T is CH₂; or
(vi) L is C, Q is CH, T is O.

In some embodiments, the compound of formula (I) is the compound of formula (II);

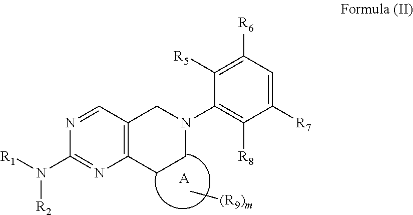

wherein, the R₁, R₂, R₅, R₆, R₇, R₈, R₉ and m have the same definition as formula (I).

In some embodiments, ring A of formula (I) is phenyl, C₅₋₆heteroaryl or C₁₀heterocyclyl, wherein the C₅₋₆heteroaryl optionally containing 1, 2 or 3 heteroatoms selected from N and S, the C₁₀heterocyclyl is a fused bicyclic which has two N atoms and one O atom in the ring.

In some embodiments, R₉ of formula (I) is selected from the group consisting of H, halogen, cyano, C₁₋₆alkyl, halogen substituted C₁₋₆alkyl, —(CH₂)ₙNR₁₁R₁₂ substituted amino, C₁₋₆alkoxy which substituted with substituted C₆ heterocyclyl, wherein R₁₁ and R₁₂ are each optionally selected from C₁₋₆alky.

In some embodiments,

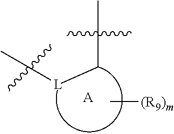

of formula (I) is

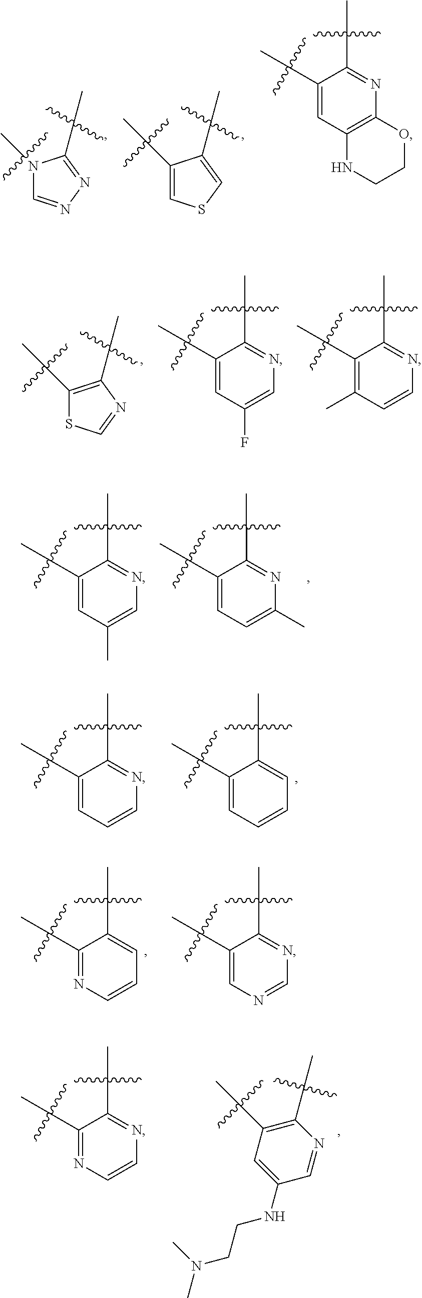

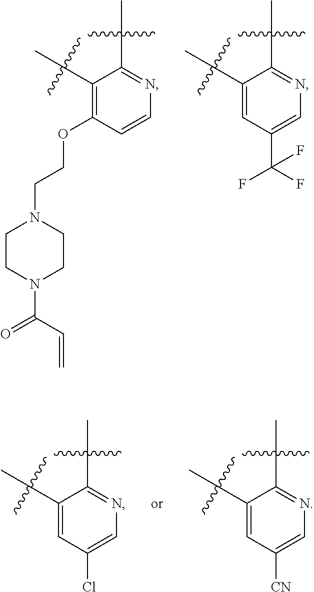 [SMILES: *c1nccc(OCCN2CCN(C(=O)C=C)CC2)c1* ; *c1cc(C(F)(F)F)cnc1* ; *c1cc(Cl)cnc1* ; *c1cc(C#N)cnc1*]

In some embodiments, R₁ of formula (I) is H, R₂ is selected from the group consisting of C₅₋₆cycloalkyl, substituted C₅₋₆cycloalkyl, C₅₋₇heterocyclyl, substituted C₅₋₇heterocyclyl and phenyl, wherein the C₅₋₇heterocyclyl optionally containing 1 or 2 heteroatoms selected from N and O.

In some embodiments, R₂ of formula (I) is substituted with 1 or 2 R₁₃ substituents, R₁₃ is selected from the group consisting of C₅₋₆heterocyclyl, substituted C₅₋₆heterocyclyl, —NR₁₄R₁₅ and —CO—R₁₆, R₁₄ is H, R₁₅ is —CO—C₂₋₄alkenyl, R₁₆ is C₁₋₃alkyl or substituted C₁₋₃alkyl.

In some embodiments, R₁₃ of formula (I) is substituted with 0-1 R₁₈ substituent, R₁₈ is selected from the group consisting of C₁₋₆alkyl, C₅₋₆ heterocyclyl and substituted C₅₋₆ heterocyclyl, wherein the C₅₋₆ heterocyclyl containing 1 or 2 heteroatoms selected from N and O.

In some embodiments, R₂ of formula (I) is

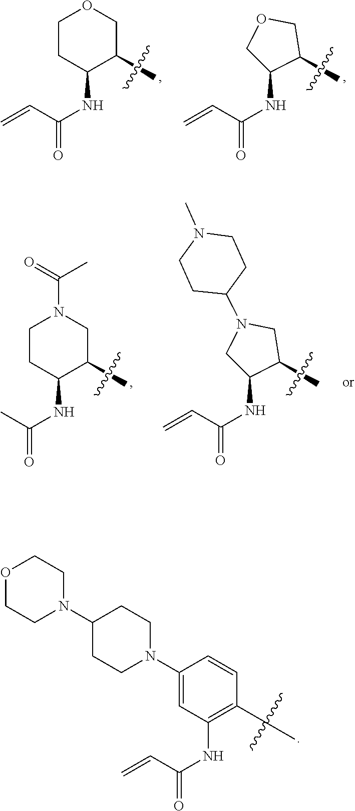

In some embodiments, R₅, R₆, R₇ and R₈ of formula (I) are each independently selected from the group consisting of hydrogen, halogen, C₁₋₃alkoxy and substituted C₁₋₃alkoxy.

In some embodiments, R₅ and R₈ of formula (I) are selected from the following groups:
(i) Both R₅ and R₈ are chlorine;
(ii) Both R₅ and R₈ are hydrogen;
(iii) R₅ is hydrogen, R₈ is chlorine; or
(iv) R₅ is chlorine, R₈ is hydrogen.

In some embodiments, R₆ and R₇ of formula (I) are selected from the following groups:
(i) Both R₁ and R₇ are methoxy;
(ii) R₆ is methoxy, R₇ is H; or
(iii) R₆ is H, R₇ is methoxy.

In some embodiments, m of formula (I) is 0 or 1.

In some embodiments, n of formula (I) is 2.

The present invention further provides a compound or a pharmaceutical acceptable salt thereof, wherein the compound is selected from the group consisting of:
(1) N-(2-((6-(2,6-dichloro-3,5-dimethoxyphenyl)-5,6-dihydropyrimido[5,4-c][1,8]naphthyridin-2-yl)amino)-5-(4-morpholinopiperidin-1-yl)phenyl)acrylamide,
(2) N-((3S,4S)-3-((6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide,
(3) N-((3S,4S)-3-((6-(2,6-dichloro-3,5-dimethoxyphenyl)-5-oxo-5,6-dihydropyrimido [5,4-c][1,8]naphthyridin-2-yl)amino)tetrahydro-2H-pyran-4-yl)acrylamide;
(4) N-((3S,4S)-3-((6-(2,6-dichloro-3,5-dimethoxyphenyl)-5-methyl-5,6-dihydropyrimido[5,4-c][1,8]naphthyridin-2-yl)amino)tetrahydro-2H-pyran-4-yl)acrylamide;
(5) N-((3S,4S)-3-((6-(2,6-dichloro-3,5-dimethoxyphenyl)-5,6-dihydropyrimido[5,4-e][1,2,4]triazolo[4,3-a]pyrimidin-2-yl)amino)tetrahydro-2H-pyran-4-yl)acrylamide,
(6) N-((3S,4S)-3-((6-(2,6-dichloro-3,5-dimethoxyphenyl)-5,6-dihydropyrimido[5,4-c]quinolin-2-yl)amino)tetrahydro-2H-pyran-4-yl)acrylamide;
(7) N-((3R,4S)-4-((6-(2,6-dichloro-3,5-dimethoxyphenyl)-5,6-dihydropyrimido[5,4-c][1,8]naphthyridin-2-yl)amino)tetrahydrofuran-3-yl)acrylamide;
(8) N-((3S,4S)-3-((6-(2,6-dichloro-3,5-dimethoxyphenyl)-5,6-dihydropyrimido[5,4-c][1,5]naphthyridin-2-yl)amino)tetrahydro-2H-pyran-4-yl)acrylamide;
(9) N-((3S,4S)-3-((6-(2,6-dichloro-3,5-dimethoxyphenyl)-5,6-dihydropyrido[2,3-d:4,5-d′]dipyrimidin-2-yl)amino)tetrahydro-2H-pyran-4-yl)acrylamide;
(10) N-((3S,4S)-3-((6-(2,6-dichloro-3,5-dimethoxyphenyl)-5,6-dihydropyrazino[2′,3′:5,6]pyrido[4,3-d]pyrimidin-2-yl)amino)tetrahydro-2H-pyran-4-yl)acrylamide;
(11) N-((3S,4S)-3-((6-(2,6-dichloro-3,5-dimethoxyphenyl)-9-fluoro-5,6-dihydropyrimido[5,4-c][1,8]naphthyridin-2-yl)amino)tetrahydro-2H-pyran-4-yl)acrylamide;
(12) N-((3S,4S)-3-((6-(2,6-dichloro-3,5-dimethoxyphenyl)-10-methyl-5,6-dihydropyrimido[5,4-c][1,8]naphthyridin-2-yl)amino)tetrahydro-2H-pyran-4-yl)acrylamide,
(13) N-((3S,4S)-3-((6-(2,6-dichloro-3,5-dimethoxyphenyl)-9-methyl-5,6-dihydropyrimido[5,4-c][1,8]naphthyridin-2-yl)amino)tetrahydro-2H-pyran-4-yl)acrylamide;
(14) N-((3S,4S)-3-((6-(2-chloro-3,5-dimethoxyphenyl)-9-methyl-5,6-dihydropyrimido[5,4-c][1,8]naphthyridin-2-yl)amino)tetrahydro-2H-pyran-4-yl)acrylamide;
(15) N-((3S,4S)-3-((6-(2,6-dichloro-3,5-dimethoxyphenyl)-8-methyl-5,6-dihydropyrimido[5,4-c][1,8]naphthyridin-2-yl)amino)tetrahydro-2H-pyran-4-yl)acrylamide;
(16) N-((3S,4S)-3-((6-(2,6-dichloro-3,5-dimethoxyphenyl)-9-(trifluoromethyl)-5,6-dihydropyrimido[5,4-c][1,8]naphthyridin-2-yl)amino)tetrahydro-2H-pyran-4-yl)acrylamide;
(17) N-((3S,4S)-3-((6′-(2,6-dichloro-3,5-dimethoxyphenyl)-6′H-spiro[cyclopropane-1,5′-pyrimido[5,4-c][1,8]naphthyridin]-2′-yl)amino)tetrahydro-2H-pyran-4-yl)acrylamide;
(18) N-((3S,4S)-3-((6-(2,6-dichloro-3,5-dimethoxyphenyl)pyrimido[4,5-f][1,7]naphthyridin-2-yl)amino)tetrahydro-2H-pyran-4-yl)acrylamide;
(19) N-((3S,4S)-3-((6-(2,6-dichloro-3,5-dimethoxyphenyl)-5-methyl-5,6-dihydrothieno[3′,4′:5,6]pyrido[4,3-d]pyrimidin-2-yl)amino)tetrahydro-2H-pyran-4-yl)acrylamide;
(20) N-((3S,4S)-3-((6-(2,6-dichloro-3,5-dimethoxyphenyl)-6H-pyrido[3′,2′:4,5]pyrano[3,2-c]pyrimidin-2-yl)amino)tetrahydro-2H-pyran-4-yl)acrylamide;
(21) N-((3S,4S)-3-((6-(2-chloro-5-methoxyphenyl)-5,6-dihydropyrimido[5,4-c][1,8]naphthyridin-2-yl)amino)tetrahydro-2H-pyran-4-yl)acrylamide,
(22) N-(3S,4S)-3-((6-(2,6-dichloro-3,5-dimethoxyphenyl)-6,9,10,11-tetrahydro-5H-[1,4]oxazino[2,3-b]pyrimido[4,5-f][1,8]naphthyridin-2-yl)amino)tetrahydro-2H-pyran-4-yl)acrylamide;
(23) N-((3R,4S)-4-((10-(2-(4-acryloylpiperazin-1-yl)ethoxy)-6-(2,6-dichloro-3,5-dimethoxyphenyl)-5,6-dihydropyrimido[5,4-c][1,8]naphthyridin-2-yl)amino)tetrahydrofuran-3-yl)acrylamide;
(24) N-((3R,4S)-1-acetyl-3-((6-(2,6-dichloro-3,5-dimethoxyphenyl)-5,6-dihydropyrimido[5,4-c][1,8]naphthyridin-2-yl)amino)piperidin-4-yl)acrylamide,
(25) N-((3S,4S)-3-((4-(2,6-dichloro-3,5-dimethoxyphenyl)-5-methyl-4,5-dihydrothiazolo[5′,4′:5,6]pyrido[4,3-d]pyrimidin-8-yl)amino)tetrahydro-2H-pyran-4-yl)acrylamide;
(26) N-((3S,4S)-3-((6-(2,6-dichloro-3,5-dimethoxyphenyl)-9-((2-(dimethylamino)ethyl) amino)-5,6-dihydropyrimido[5,4-c][1,8]naphthyridin-2-yl)amino)tetrahydro-2H-pyran-4-yl)acrylamide;
(27) N-((3S,4R)-4-((6-(2,6-dichloro-3,5-dimethoxyphenyl)-5,6-dihydropyrimido[5,4-c][1,8]naphthyridin-2-yl)amino)-1-(1-methylpiperidin-4-yl)pyrrolidin-3-yl)acrylamide;
(28) N-((3S,4S)-3-((9-chloro-6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide,
(29) N-((3S,4S)-3-((9-cyano-6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide,
(30) N-((3S,4S)-3-((6-(2-chloro-5-methoxyphenyl)-9-methyl-5,6-dihydropyrimido[5,4-c][1,8]naphthyridin-2-yl)amino)tetrahydro-2H-pyran-4-yl)acrylamide;
(31) N-((3S,4S)-3-((6-(2-chloro-3-methoxyphenyl)-9-methyl-5,6-dihydropyrimido[5,4-c][1,8]naphthyridin-2-yl)amino)tetrahydro-2H-pyran-4-yl)acrylamide;
(32) N-((3S,4S)-3-((6-(2,6-dichloro-3-methoxyphenyl)-9-methyl-5,6-dihydropyrimido[5,4-c][1,8]naphthyridin-2-yl)amino)tetrahydro-2H-pyran-4-yl)acrylamide;
(33) N-((3S,4S)-3-dichloro-3,5-dimethoxyphenyl)-[1,2,4]triazolo[4′,3′:1,6]pyrido[2,3-d]pyrimidin-2-yl)amino)tetrahydro-2H-pyran-4-yl)acrylamide; or
(34) N-(3-((9-chloro-6-(2,6-dichloro-3,5-dimethoxyphenyl)-5-oxo-5,6-dihydropyrimido[5,4-c][1,8]naphthyridin-2-yl)amino)tetrahydro-2-pyran-4-yl)acrylamide.

The present invention also provides a pharmaceutical composition comprising a therapeutically effective amount of at least any one of the compounds of formula (I) and at least one pharmaceutically acceptable excipient.

The present invention further provides a pharmaceutically composition in which the weight ratio of the compound of formula (I) and the said excipient is 0.0001-10.

The present invention provides the use of the compound of formula (I) or pharmaceutical composition in the manufacture of a medicament.

The present invention further provides a preferred technical solution for such use:

Preferably, the use is for treating, preventing, delaying or arresting the onset or progression of cancer or cancer metastasis.

Preferably, the use is for manufacturing a medicament for use in the treatment of the disease mediated by FGFR4.

Preferably, the disease is cancer.

Preferably, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, Hepatocellular carcinoma, head and neck tumors, hepatobiliary cell carcinoma, myelodysplastic syndrome, malignant glioma, prostate cancer, thyroid cancer, Schwann cell tumor, lung squamous cell carcinoma, lichenoid keratosis, Synovial sarcoma, skin cancer, pancreatic cancer, testicular cancer or liposarcoma.

Preferably, the use is as a FGFR4 inhibitor.

The present invention also provides a method of treating or preventing the disease mediated by FGFR4 by administering a therapeutically effective amount at least any one of the compounds of Formula (I) or the pharmaceutical composition to a subject.

Preferably, in the above method, the disease mediated by FGFR4 is cancer.

Preferably, in the above method, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, Hepatocellular carcinoma, head and neck tumors, hepatobiliary cell carcinoma, myelodysplastic syndrome, malignant glioma, prostate cancer, thyroid cancer, Schwann cell tumor, lung squamous cell carcinoma, lichenoid keratosis, Synovial sarcoma, skin cancer, pancreatic cancer, testicular cancer or liposarcoma.

The present invention also provides a method for treating cancer, which comprises administrating at least any one of the compounds of Formula (I) or the pharmaceutical composition to a subject, the said cancer is breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, Hepatocellular carcinoma, head and neck tumors, hepatobiliary cell carcinoma, myelodysplastic syndrome, malignant glioma, prostate cancer, thyroid cancer, Schwann cell tumor, lung squamous cell carcinoma, lichenoid keratosis, Synovial sarcoma, skin cancer, pancreatic cancer, testicular cancer or liposarcoma.

Preferably, in the above method, the subject is human.

Unless otherwise stated, the terms used in the present invention have the following meanings:

The term “alkyl” includes saturated hydrocarbon groups having straight and branched-chain or cyclic moieties. For example, alkyl group includes but not limited to methyl, ethyl, propyl, isopropyl, cyclopropyl, n-butyl, isobutyl, sec-butyl, t-butyl, cyclobutyl, n-pentyl, 3-(2-methyl)butyl, 2-pentyl, 2-methylbutyl, neopentyl, cyclopentyl, n-hexyl, 2-hexyl, 2-methylpentyl and cyclohexyl. Similarly, C₁₋₈, as in C₁₋₈ alkyl is defined to identify the group as having 1, 2, 3, 4, 5, 6, 7 or 8 carbon atoms in a linear, branched or cyclic arrangement.

“Alkenyl” and “alkynyl” groups include straight, branched-chain or cyclic alkenes and alkynes. Likewise, “C₂₋₈ alkenyl” and “C₂₋₈ alkynyl” means an alkenyl or alkynyl group having 2, 3, 4, 5, 6, 7 or 8 carbon atoms in linear or branched-chain or cyclic arrangement.

“Alkoxy” refers to the oxygen ethers form of the previously described straight or branched-chain or cyclic alkyl groups.

The term “aryl”, as used herein, unless otherwise indicated, refers to an unsubstituted or substituted mono- or polycyclic ring system containing carbon atoms. The preferred aryl is 6-10 membered mono- or bi-cyclic aromatic ring systems. Phenyl and naphthyl are preferred aryls. The most preferred aryl is phenyl.

The term “heteroaryl” refers to a monovalent heteroatom group formed by the removal of one hydrogen atom from a carbon atom of a parent heteroaromatic ring system. The heteroaryl group includes a 5- to 7-membered aromatic, monocyclic ring comprising at least one hetero atom selected from N, O or S, for example, 1 to 4 hetero atoms, or preferably 1 to 3 hetero atoms, and the other atom on the ring is carbon; the polyheteroaryl ring includes at least one hetero atom selected from N, O or S, for example, 1 to 4 hetero atoms, or preferably 1 to 3 hetero atoms, and other atoms on the ring is carbon and at least one of the heteroatoms is on the aromatic ring Particularly preferred heteroaryl groups are C₃₋₁₀ heteroaryl groups including, but not limited to, pyrrolyl, furyl, thienyl, pyridyl, pyranyl, pyrazolyl, pyrimidinyl, pyridazinyl, pyrazinyl, imidazolyl, thiazolyl, oxazolyl, isoxazoyl, triazolyl, indolyl, benzofuranyl, benzothiazolyl, benzimidazolyl, benzopyrazolyl, benzotriazolyl, carbazolyl, quinolyl, isoquinolinyl, purinyl and the similar groups.

However, in any case, the heteroaryl group and the aryl group do not cross each other or contain each other. Thus, according to the above definition, if at least one all-carbon aromatic ring is fused to a heterocyclic group, a heteroaryl group is obtained instead of an aryl group.

“Cycloalkyl” refers to a saturated or unsaturated cyclic group without aromaticity. According to the particular level of saturation, the terms “cycloalkyl”, “cycloalkenyl” or “cycloalkynyl” are employed, respectively. Representative cycloalkyl groups include, but not limited to, cyclopropane, cyclobutane, cyclopentane, cyclohexane or cyclohexene, and the like. Specifically, the cycloalkyl group may be a C₃₋₁₀ cycloalkyl group such as a C₃₋₆ cycloalkyl group.

“Heterocyclyl” refers to a saturated or unsaturated cyclic group without aromaticity, and wherein one or more carbon atoms (and the attached hydrogen atoms) can be substituted with the same or different heteroatom and the corresponding hydrogen atom, respectively. Representative heteroatom which substitute the carbon atoms include, but are not limited to, N, P, O, S, and Si. The terms “heterocycloalkyl” or “heterocyclenyl” are used, respectively when it is necessary to describe the particular degree of saturation. Representative heterocyclyl groups include, but are not limited to, epoxy compounds, imidazolidines, morpholine, piperazine, piperidine, pyrazolidine, pyrrolidine, quinuclidine, tetrahydrofuran or tetrahydropyran, and the like. The substituted heterocyclyl group also includes a ring system substituted with at least one oxygen-containing (═O) or oxide (—O—) substituent, such as piperidine-nitrogen-oxide, morpholinyl-nitrogen-oxide, 1-oxo-1-thiomorpholinyl and 1-dioxy-1-thiomorpholinyl.

However, in any case, the heterocycloalkyl group and the cycloalkyl group do not cross each other or contain each other. Thus, according to the above definition, if at least one carbocyclic ring is fused to a heterocycloalkyl group to form a di-, poly- or spiro-ring, it will still be defined as a heterocycloalkyl group.

Besides, if a heteroaryl is fused to a heterocyclyl to form a di-, poly- or spiro-ring, it will be defined as a heterocyclyl instead of heteroaryl.

“Halogen” refers to fluorine (F), chlorine (Cl), bromine (Br) or iodine (I). Preferred halogen refers to fluorine, chlorine and bromine.

“Halo” refers to a fluoro, chloro, bromo or iodo group. Preferred halo refers to fluoro and chloro.

“Substituted” refers that one or more hydrogen atoms in a group are each substituted with the same or different substituents. Representative substituents include, but are not limited to, halogen, amino, hydroxy, oxo, carbonyl, cyano, alkyl, alkoxy, aryl, cycloalkyl, heterocyclyl, heteroaryl, alkylpiperazine, morpholinyl. In some embodiments, the substituents include, but are not limited to, F, Cl, CN, amino, hydroxyl, cyano, methy, trifluoromethy, cyclopropyl, phenyl, dimethylamino,

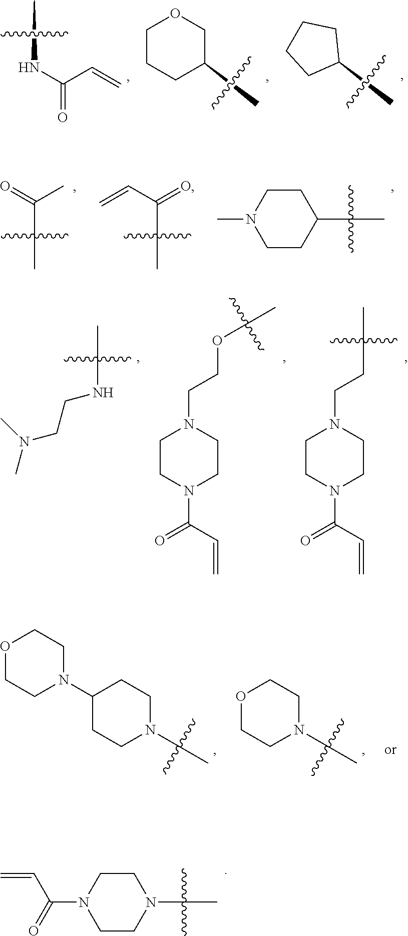

Whenever, the term “alkyl” or “aryl” or its prefix root appear in the name of a substituent (such as an aralkyl group, or a dialkylamino group), the substituents should be defined according to the aforementioned “alkyl” and “aryl”. The specified number of carbon atoms (e.g., C₁₋₆) will independently represent the number of carbon atoms in an alkyl moiety or an alkyl moiety in a larger substituent (wherein the alkyl group is the prefix root).

“Compound” as used herein includes a compound of Formula (I), and all pharmaceutically acceptable forms thereof. These pharmaceutically acceptable forms include salts, solvates, non-covalent complexes, chelates or prodrugs thereof, or any mixture of all of the above.

“Pharmaceutically acceptable” means it is well-known for use in animals, particularly for use in humans.

The term “composition” as used in the present invention includes products comprising a specific amount of a particular component, as well as any product derived directly or indirectly from a particular quantity of a particular component. Therefore, a pharmaceutical composition comprising the compound of the present invention as an active ingredient and a method of preparing the same are the contents of the present invention.

“Therapeutically effective amount” means that when a compound is administered to a subject to treat and prevent and/or inhibit at least one clinical condition of a disease, condition, symptom, indication, and/or discomfort, a dose sufficient to produce a certain effect on the treatment of disease, condition, symptom, indication, or discomfort. The specific “effective therapeutic amount” may vary depending on the compound, the route of administration, the age of the patient, the weight of the patient, the type of the disease or discomfort being treated, the symptoms and severity, and the like. Wherever possible, a suitable dosage will be apparent to those skilled in the art and may be determined by routine experimentation.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicyclic, saccharinic or trifluoroacetic. Pharmaceutically acceptable basic/cationic salts include and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

When the compound of Formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium, and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procacine, purines, theobromine, tri ethyl amine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred acids are citric, hydrobromic, formic, hydrochloric, maleic, phosphoric, sulfuric and tartaric acids. Particularly preferred acids are formic and hydrochloric acid. Since the compounds of Formula (I) are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administrated. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical composition of this invention may include a pharmaceutically acceptable carrier and a compound, or a pharmaceutically acceptable salt, of Formula I. The compounds of Formula I, or a pharmaceutically acceptable salt thereof, can also be included in pharmaceutical compositions in combination with one or more other therapeutically active compounds.

The pharmaceutical carrier employed can be, for example, a solid, liquid, or gas Examples of solid carriers include lactose, gypsum powder, sucrose, talc, gelatin, agar, pectin, acacia, magnesium stearate, stearic acid, mannitol, sorbitol, microcrystalline cellulose, inorganic salts, starch, pregelatinized starch, powdered sugar and the like. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets Because of their ease of administration, tablets and capsules are the preferred oral dosage units whereby solid pharmaceutical carriers are employed. Optionally, tablets may be coated by standard aqueous or nonaqueous techniques.

A tablet containing the compounds or composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a lubricant, inert diluent, surface active or dispersing agent. Molded tablets may be made by molding in a suitable machine, a mixture of the powdered compound or composition moistened with an inert liquid diluent. Each tablet preferably contains from about 0.01 mg to about 5 g of the active ingredient and each cachet or capsule preferably containing from about 0.1 mg to about 0.5 g of the active ingredient. For example, a formulation intended for the oral administration to humans may contain from about 0.1 mg to about 0.5 g of active agent, compounded with an appropriate and convenient amount of carrier material which may vary from about 5 to about 99.99 percent of the total composition. Unit dosage forms will generally contain between from about 0.1 mg to about 0.5 g of the active ingredient, typically 0.1 mg, 0.2 mg, 0.5 mg, 1 mg, 2 mg, 2.5 mg, 5 mg, 10 mg, 25 mg, 50 mg, 100 mg, 200 mg, 300 mg, 400 mg or 500 mg.

Pharmaceutical compositions of the present invention suitable for parenteral administration may be prepared as solutions or suspensions of the active compounds in water. A suitable surfactant can be included such as sodium lauryl sulfate, polysorbate-80 (Tween-80), polyoxyethylene hydrogenated castor oil, poloxamer. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacterial and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 50 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carriers followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or a pharmaceutically acceptable salts thereof, may also be prepared in powder or liquid concentrate form.

### EXAMPLES

To be clearer, the present invention is further exemplified by the following examples. The following examples are intended to illustrate the specific embodiments of the present invention to make those skilled in art understand the present invention, but not to limit the protection scope of the present invention. In the examples of the present invention, the techniques or methods, unless expressly stated otherwise, are conventional techniques or methods in the art.

Unless otherwise indicated, all parts and percentages of the invention are calculated by weight, the temperatures are given in degrees Celsius (° C.).

The following abbreviations have been used in the examples.

ATP: Adenine nucleoside triphosphate;

(BOC)₂O: Di-tert-butyl dicarbonate;

DCM: Dichloro methane;

DIPEA: N,N-diisopropylethylamine;

DMAP: 4-dimethylaminopyridine;

DMF: N,N-dimethylformamide;

DMSO: Dimethy sulfoxide,

EA: Ethyl acetate;

HCOOEt: Ethyl formate;

HOAc: Acetic acid;

KOAc: Potassium acetate;

LCMS or LC-MS: Liquid Chromatograph Mass Spectrometer;

m-CPBA or mCPBA: M-chloroperoxybenzoic acid;

MeMgBr: Methy Magnesium Bromide;

MeOH: Methanol;

PdCl₂(dppf)CH₂Cl₂: [1,1′-Bis(diphenylphosphine)ferrocene]dichloro palladium dichloro methane complex;

Pd(OAc)₂: Palladium(II) acetate;

Pd(PPh₃)₄: Tetra (triphenylphosphine)palladium;

rt, r.t. or RT: Room temperature;

h, hr or hrs: hour;

TEA: Triethylamine;

THF: Tetrahydrofuran;

TLC: Thin layer chromatography;

Xant-phos: 4,5-Bis(diphenylphosphino)-9,9-dimethylxanthene.

### Example 1: Preparation of N-(2-((6-(2,6-dichloro-3,5-dimethoxyphenyl)-5,6-dihydropyrimido[5,4-c][1,8]naphthyridin-2-yl)amino)-5-(4-morpholinopiperidin-1-yl)phenyl)acrylamide

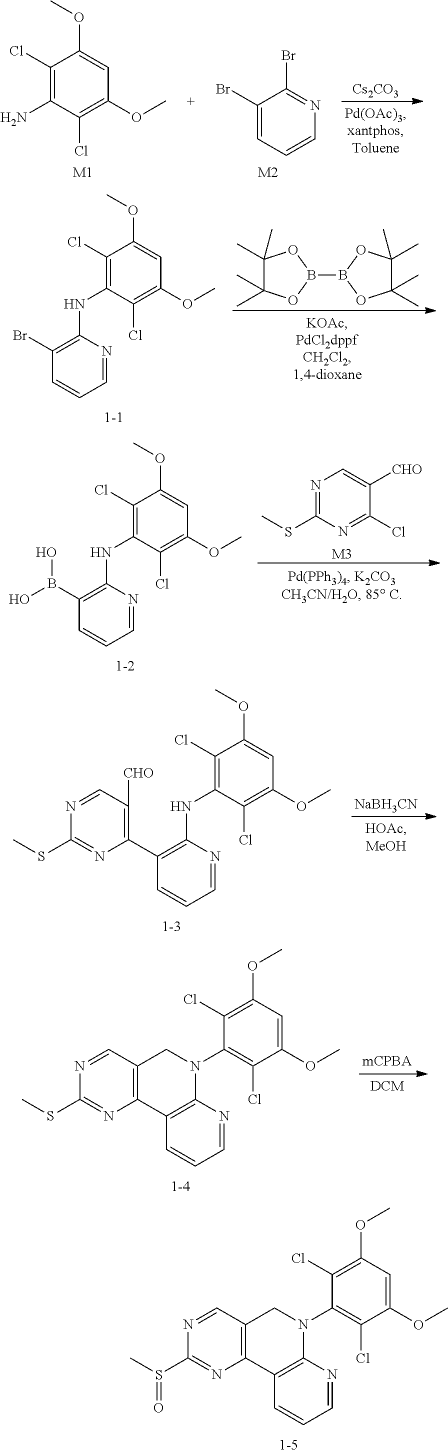

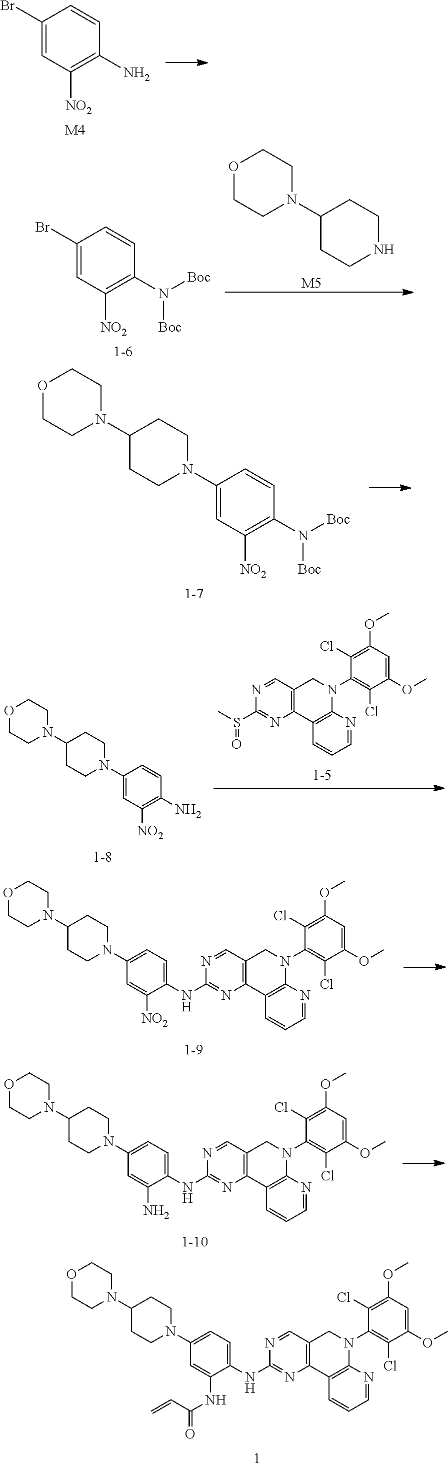

Step1: Preparation of Compound 1-1

M1 (0.85 g), M2 (0.91 g), Cs₂CO₃ (2.49 g), Pd(OAc)₂ (0.09 g), Xant-phos (0.44 g) were dissolved in toluene (40 mL), and reacted at 115° C. for 5 hrs under N₂ protection. LCMS showed the reaction was completed Concentrated under reduced pressure, the residue was dissolved in water, extracted with DCM, the organic layer was washed with water, saturated brine successively, concentrated, the residue was purified by column chromatography eluting with hexane:dichloromethane=1:5 to obtain 1.28 g compound 1-1 as a yellow solid.

LC-MS [M+H⁺] 376.9.

Step2: Preparation of Compound 1-2

Compound 1-1 (1.25 g), pinacol diborate (0.91 g), KOAc (1.20 g) and PdCl₂(dppf)CH₂Cl₂ (0.14 g) were dissolved in 1,4-dioxane (40 mL), and reacted at 100° C. for 15 hrs under N₂ protection. LCMS showed the reaction was completed. Concentrated under reduced pressure, the residue was dissolved in water, extracted with EA, the organic layer was washed with water, saturated brine successively, concentrated, the residue was purified by column chromatography eluting with dichloromethane:methonal=30:1 to obtain 1.07 g compound 1-2 as a brown solid.

LC-MS [M+H⁺] 343.0.

Step3: Preparation of Compound 1-3

Compound 1-2 (1.03 g), M3 (0.57 g), K₂CO₃ (0.83 g) and Pd(PPh₃)₄ (0.35 g) were dissolved in 35 mL of acetonitrile/12 mL of water, and reacted at 85° C. for 4.5 hrs under N₂ protection. LCMS showed the reaction was completed. Concentrated under reduced pressure, the residue was dissolved in water, extracted with DCM, the organic layer was washed with water, saturated brine successively, concentrated, the residue was purified by column chromatography eluting with dichloromethane:methonal=200:1 to obtain compound 1-3 (0.93 g) as a yellow solid.

LC-MS [M+H⁺] 451.0.

Step4: Preparation of Compound 1-4

Compound 1-3 (0.92 g), acetic acid (0.19 g) and cyano sodium borohydride (0.20 g) were dissolved in methanal (25 mL), and reacted at room temperature for 14 hrs under N₂ protection. LCMS showed the reaction was completed. Concentrated under reduced pressure, the residue was dissolved in water, saturated Na₂CO₃ aqueous solution and DCM, extracted with DCM twice, the organic layer was combined and washed with water and saturated brine successively, concentrated, the residue was purified by column chromatography eluting with DCM:CH₃OH=100:1 to obtain compound 1-4 (0.39 g) as a yellow solid.

LC-MS [M+H⁺] 434.9.

Step5: Preparation of Compound 1-5

Compound 1-4 (0.35 g) was dissolved in DCM (25 mL), mCPBA (85%) (0.36 g) was added slowly under an ice-water bath condition, after the addition, warmed to room temperature naturally and reacted for 7 hrs. LCMS showed the reaction was completed. The reaction solution was washed with saturated NaHCO₃ aqueous solution twice, the organic layers were combined, washed with water and saturated brine, dried over anhydrous Na₂SO₄ for 1 hr, filtered and concentrated under reduced pressure to obtain compound 1-5 (0.39 g) as an orange-yellow solid.

LC-MS [M+H⁺] 451.0.

Step6: Preparation of Compound 1-6

To a solution of compound M4 (10.85 g), DMAP (1.22 g), triethylamine (17.4 mL) in tetrahydrofuran (250 mL), (Boc)₂O (24.01 g) dissolved in tetrahydrofuran (40 mL) was added slowly under stirring, after the addition, the reaction was refluxed for 16 hrs under N₂ protection. TLC showed the reaction was almost completed. Concentrated under reduced pressure, the residue was dissolved in water, extracted with DCM, the organic was washed with water, saturated brine successively, concentrated, the residue was purified by column chromatography eluting with hexane:ethyl acetate=5:1 to obtain compound 1-6 (20.18 g) as a yellow solid.

LC-MS [M+H⁺] 417.1.

Step7: Preparation of Compound 1-7

Compound 1-6 (3.02 g), M5 (1.13 g), Cs₂CO₃ (5.36 g), Pd₂dba₃ (0.60 g), Xant-phos (0.76 g) were dissolved in toluene (50 mL), and reacted at 110° C. for 14 hrs under N₂ protection LCMS showed the reaction was completed. Concentrated under reduced pressure, the residue was dissolved in water, extracted with DCM, the organic was washed with water, saturated brine successively, concentrated, the residue was purified by column chromatography eluting with DCM:MeOH=40:1 to obtain compound 1-7 (2.20 g) as a red-brown sticky substance.

LC-MS [M+H⁺] 507.1.

Step8: Preparation of Compound 1-8

To a solution of compound 1-7 (2.20 g) in DCM (30 mL), trifluoroacetate (20 mL) was added slowly, reacted at room temperature for 2 hrs, the pH of the residue was adjusted to 8˜9 using saturated Na₂CO₃ aqueous solution, extracted with DCM, washed with water and saturated brine, the organic layer was dried over anhydrous Na₂SO₄ for 1 hr, filtered and concentrated under reduced pressure to obtain crude product (1.20 g) as red-brown solid which was used for the next step directly without purification.

LC-MS [M+H⁺] 307.1.

Step9: Preparation of Compound 1-9

Compound 1-8 (307 mg), compound 1-5 (451 mg) were dissolved in anhydrous DMF (10 mL), potassium tert-butoxide (169 mg) was added slowly at −10° C., after the addition, the mixture was reacted for 2 hrs after warming to room temperature naturally. LCMS showed the reaction was completed. The reaction was quenched with water, extracted with EA, the organic layer was washed with water and saturated brine successively, concentrated, the residue was purified by column chromatography eluting with dichloromethane:methanol=40:1 to obtain compound 1-9 (200 mg) as a yellow solid.

LC-MS [M+H⁺] 693.1.

Step10: Preparation of Compound 1-10

The solution of compound 1-9 (0.20 g), reduced iron powder (0.13 g) and ammonium chloride (0.13 g) in ethanol (30 mL) and water (6 mL) was refluxed for 3 hrs under N₂ protection. LCMS showed the reaction was completed. The reaction mixture was filtered without cooling, filtrate was concentrated under reduced pressure, the residue was purified by column chromatography eluting with DCM:MeOH=9:1 to obtain compound 1-10 (0.16 g) as an orange-yellow solid.

LC-MS [M+H⁺] 663.1.

Step11: Preparation of Compound 1

To a solution of compound 1-10 (100 mg) in DCM (8 mL) was added trimethylamine (0.1 mL), and then acrylic chloride was added slowly at −10° C., the reaction was reacted at such temperature for 1 h. LCMS showed the reaction was almost completed, the mixture was quenched with saturated NaHCO₃ aqueous solution, the DCM layer was washed with water and saturated brine successively, concentrated under reduced pressure, the residue was purified by preparative TLC eluting with DCM:MeOH=10:1 to obtain compound 1 (43 mg) as an orange-yellow solid.

LC-MS [M+H⁺] 717.2.

### Example 2: Preparation of N-((3S,4S)-3-((6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide

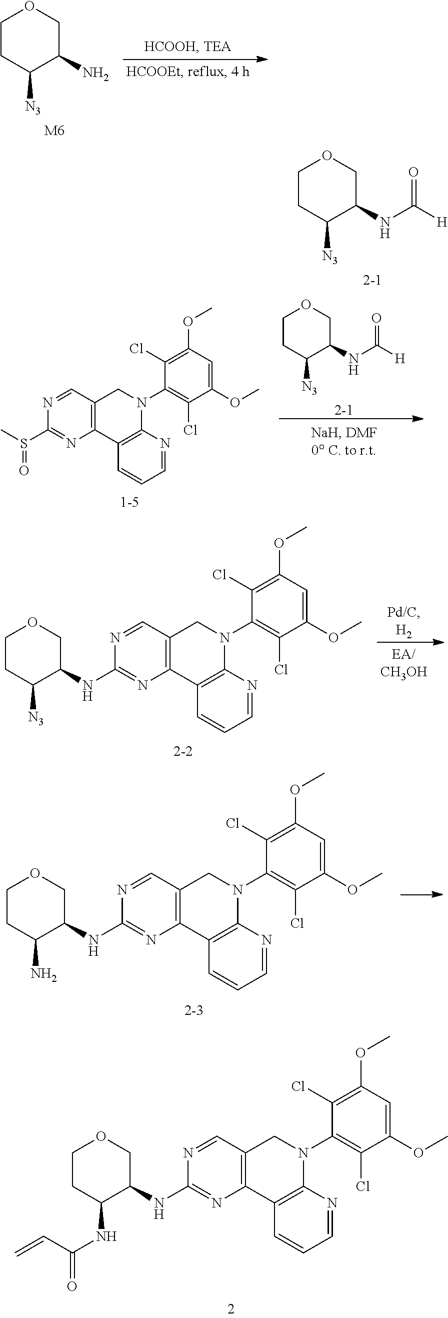

Step1: Preparation of Compound 2-1

M6 (3.57 g), TEA (6.2 mL) and formic acid (88%)(2.09 g) were dissolved in ethyl formate (100 mL), the reaction was refluxed for 4 hrs. TLC showed the reaction was completed. Concentrated under reduced pressure, the residue was dissolved in ethyl acetate, the organic layer was washed with a little water and saturated brine successively, concentrated to obtain compound 2-1 (3.49 g) as an off-white solid which was used in the next reaction directly without purification

Step2: Preparation of Compound 2-2

To a solution of compound 2-1 (90 mg) in anhydrous DMF (2 mL), NaH (60%) (42 mg) was added slowly under an ice-salt bath condition, the reaction was reacted at such temperature for 40 mins, and then compound 1-5 (239 mg) was added, after the addition, the reaction was warmed to rt and reacted for 40 mins. LCMS showed the reaction was completed. The reaction mixture was quenched with water, the organic layer was washed with water and saturated brine successively, concentrated, the residue was purified by column chromatography eluting with DCM:CH₃OH=40:1 to obtain compound 2-2 (139 mg) as an orange-yellow sticky substance.

LC-MS [M+H⁺] 529.1.

Step3: Preparation of Compound 2-3

A mixture of compound 2-2 (135 mg) and 10% Pd/C (50 mg) in EA/CH₃OH (15 mL/10 mL), was reacted at 30° C. for 2 hrs under H₂ condition. Filtered, concentrated under reduced pressure to obtain crude product of compound 2-3 (132 mg) as an orange-yellow sticky substance which was used in the next reaction directly without purification.

LC-MS [M+H⁺] 503.1.

Step4: Preparation of Compound 2

To a solution of compound 2-3 (130 mg) in DCM (8 mL), trimethylamine (0.12 mL) was added, and acrylic chloride (23 mg) was added slowly at −20° C., the reaction was reacted at such temperature for 1 h. LCMS showed the reaction was completed, quenched with saturated NaHCO₃ aqueous solution, the DCM layer was washed with water and saturated brine successively, concentrated under reduced pressure, the residue was purified by thick preparative TLC eluting with DCM/MeOH=40.1 to obtain compound 2 (41 mg) as a light yellow solid.

LC-MS [M+H⁺] 557.1.

### Example 3: Preparation of N-((3S,4S)-3-((6-(2,6-dichloro-3,5-dimethoxyphenyl)-5-oxo-5,6-dihydropyrimido[5,4-c][1,8]naphthyridin-2-yl)amino)tetrahydro-2H-pyran-4-yl)acrylamide

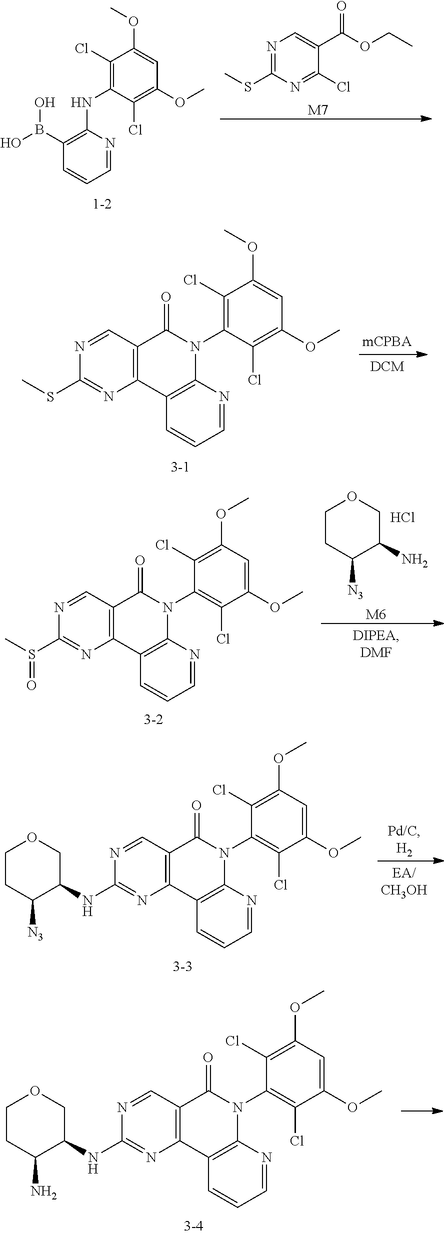

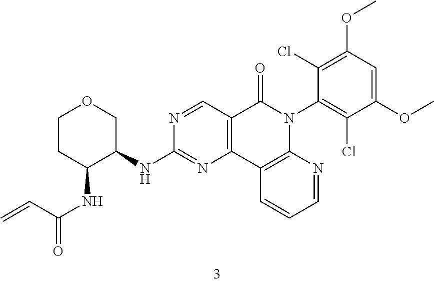 [SMILES: C=CC(=O)N[C@H]1CCOC[C@H]1Nc1ncc2c(=O)n(-c3c(Cl)c(OC)cc(OC)c3Cl)c3ncccc3c2n1]

Step1: Preparation of Compound 3-1

Compound 3-1 was prepared using a similar method shown in step 3 of Example 1, 4-chloro-2-methylthiopyrimidine-5-ethyl carboxylate (M7) replaced M3 as the original material to prepared compound 3-1.

LC-MS [M+H⁺] 449.0.

Step2: Preparation of Compound 3-2

Compound 3-2 was prepared using a similar method described in step 5 of Example 1.

LC-MS [M+H⁺] 464.9.

Step3: Preparation of Compound 3-3

Compound 3-2 (780 mg), M6 (300 mg), DIPEA (1.26 mL) and DMF (8 mL) was added into the sealed tube successively, and reacted at 80° C. for 2 hrs. Quenched with water, extracted with EA, washed with saturated brine four times, concentrated, and purified by column chromatography eluting with DCM/MeOH=100.1 to obtain compound 3-3 as an orange-yellow solid.

LC-MS [M+H⁺] 543.0.

Step4: Preparation of Compound 3-4

Compound 3-4 was prepared using a similar method described in step 3 of Example 2.

LC-MS [M+H⁺] 517.1.

Step5: Preparation of Compound 3

Compound 3 was prepared using a similar method described in step4 of Example 2.

LC-MS [M+H⁺] 571.5.

### Example 4: Preparation of N-((3S,4S)-3-((6-(2,6-dichloro-3,5-dimethoxyphenyl)-5-methyl-5,6-dihydropyrimido [5,4-c][1,8]naphthyridin-2-yl)amino)tetrahydro-2H-pyran-4-yl)acrylamide

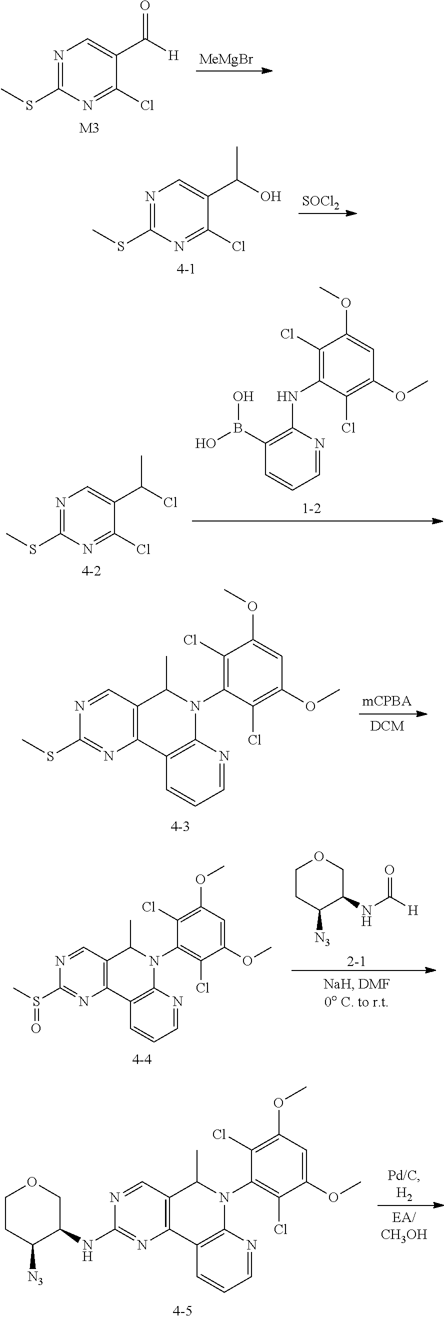

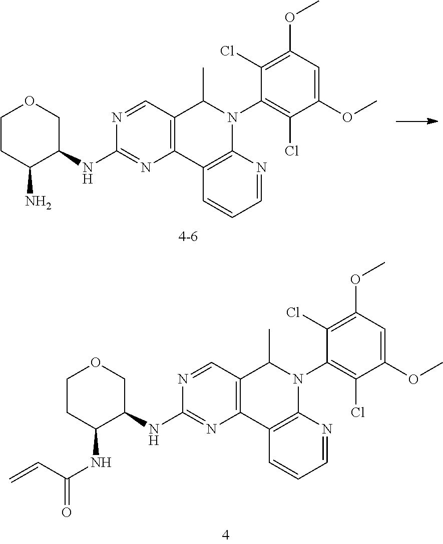

Step1: Preparation of Compound 4-1

M3 (3.77 g) was dissolved in anhydrous THF (100 mL), methyl magnesium bromide (1 M in THF) (1.2 mL) was added slowly in an ice-water bath (<5° C.) condition, and continued reacting for 1 h at the same temperature. LCMS showed the reaction was completed. The reaction was quenched with ammonium chloride aqueous solution, extracted with DCM, the organic layer was washed with water and saturated brine successively, concentrated, the residue was purified by column chromatography eluting with DCM:CH₃OH=50:1 to obtain compound 4-1 (3.72 g) as a yellow liquid.

LC-MS [M+H⁺] 205.4.

Step2: Preparation of Compound 4-2

Compound 4-1 (2.05 g) was dissolved in dichloromethane (20 mL), thionyl chloride (3.63 mL) was added slowly in an ice-water bath (<5° C.) condition, after the addition, continued reacting for 2 hrs at the same temperature. LCMS showed the reaction was completed. The reaction solution was added into a stirring ice-water mixture (1 L), extracted with dichloromethane twice, the organic layers were combined, washed with water and saturated brine successively, the residue was purified by column chromatography eluting with hexane:dichloromethane=3:1 to obtain compound 4-2 as a light yellow liquid.

LC-MS [M+H⁺] 223.3.

Step3: Preparation of Compound 4-3

Compound 4-3 was prepared using a similar method described in step 3 of Example 1, compound 4-2 replaced M3 as the original material.

LC-MS [M+H⁺] 449.0.

Step4: Preparation of Compound 4-4

Compound 4-4 was prepared using a similar method described in step 5 of Example 1.

LC-MS [M+H⁺] 465.0.

Step5: Preparation of Compound 4-5

Compound 4-5 was prepared using a similar method described in step 2 of Example 2.

LC-MS [M+H⁺] 543.0.

Step6: Preparation of Compound 4-6

Compound 4-6 was prepared using a similar method described in step 3 of Example 2.

LC-MS [M+H⁺] 517.1.

Step7: Preparation of Compound 4

Compound 4 was prepared using a similar method described in step 4 of Example 2.

LC-MS [M+H⁺] 571.1.

The compounds in Table 1 can be prepared using a similar method described in aforementioned examples with the different original materials and the appropriate reagents. For example, when M2 in Example 1 is replaced by

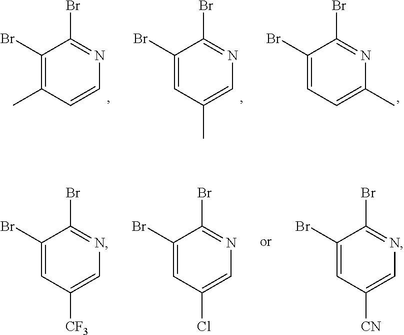

the compound 12, 13, 15, 16, 28 or 29 can be prepared with reference to the preparation method of Example 2.

TABLE 1

| Ex. | | | LC-MS |
|---|---|---|---|
| NO. | Chemical Structure | Chemical Name | [M + H⁺] |
| 5 | 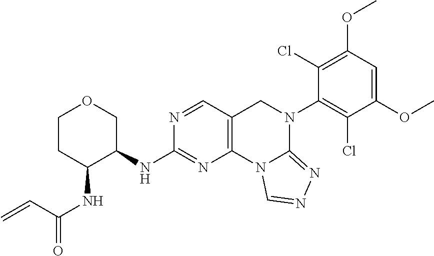 [SMILES: C=CC(=O)N[C@H]1CCOC[C@H]1Nc1ncc2c(n1)-n1cnnc1N(c1c(Cl)c(OC)cc(OC)c1Cl)C2] | N-((3S,4S)-3-((6-(2,6-dichloro-3,5-dimethoxy- phenyl)-5,6-dihydropyrimido[5,4-e][1,2,4]tria- zolo[4,3-a]pyrimidin-2-yl)amino)tetrahydro- 2H-pyran-4-yl)acrylamide | 547.1 |
| 6 | 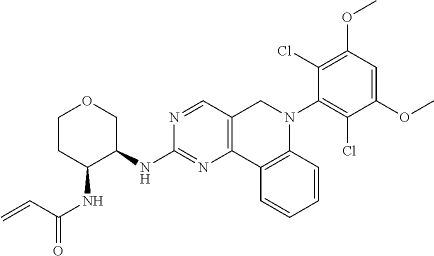 [SMILES: C=CC(=O)N[C@H]1CCOC[C@H]1Nc1ncc2c(n1)-c1ccccc1N(c1c(Cl)c(OC)cc(OC)c1Cl)C2] | N-((3S,4S)-3-((6-(2,6-dichloro-3,5-dimethoxy- phenyl)-5,6-dihydropyrimido[5,4-c]quinolin-2- yl)amino)tetrahydro-2H-pyran-4-yl)acrylamide | 556.1 |
| 7 | 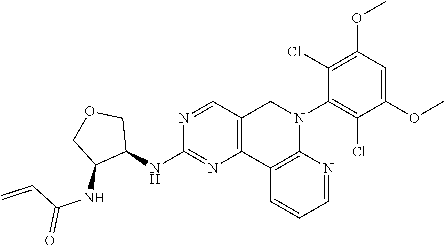 [SMILES: C=CC(=O)N[C@H]1COC[C@H]1Nc1ncc2c(n1)-c1cccnc1N(c1c(Cl)c(OC)cc(OC)c1Cl)C2] | N-((3R,4S)-4-((6-(2,6-dichloro-3,5-dimethoxy- phenyl)-5,6-dihydropyrimido[5,4-c][1,8] naphthyridin-2-yl)amino)tetrahydrofuran-3-yl)acryl- amide | 543.7 |
| 8 | 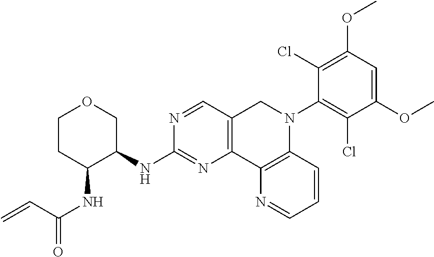 [SMILES: C=CC(=O)N[C@H]1CCOC[C@H]1Nc1ncc2c(n1)-c1ncccc1N(c1c(Cl)c(OC)cc(OC)c1Cl)C2] | N-((3S,4S)-3-((6-(2,6-dichloro-3,5-dimethoxy- phenyl)-5,6-dihydropyrimido[5,4-c][1,5] naphthyridin-2-yl)amino)tetrahydro-2H-pyran-4-yl) acrylamide | 557.1 |
| 9 | 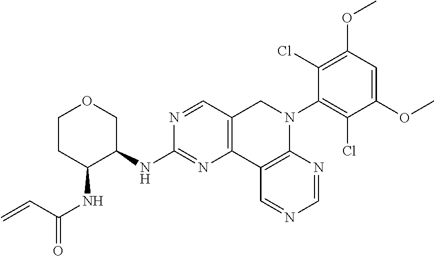 [SMILES: C=CC(=O)N[C@H]1CCOC[C@H]1Nc1ncc2c(n1)-c1cncnc1N(c1c(Cl)c(OC)cc(OC)c1Cl)C2] | N-((3S,4S)-3-((6-(2,6-dichloro-3,5-dimethoxy- phenyl)-5,6-dihydropyrido[2,3-d:4,5-d′] dipyrimidin-2-yl)amino)tetrahydro-2H-pyran-4-yl) acrylamide | 558.0 |
| 10 | 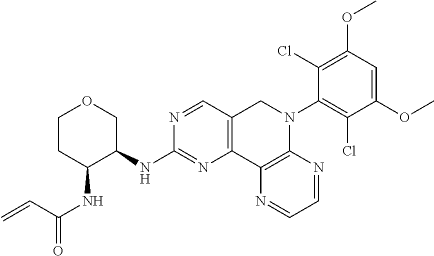 [SMILES: C=CC(=O)N[C@H]1CCOC[C@H]1Nc1ncc2c(n1)-c1nccnc1N(c1c(Cl)c(OC)cc(OC)c1Cl)C2] | N-((3S,4S)-3-((6-(2,6-dichloro-3,5-dimethoxy- phenyl)-5,6-dihydropyrazino[2′,3′:5,6]pyrido [4,3-d]pyrimidin-2-yl)amino)tetrahydro-2H- pyran-4-yl)acrylamide | 558.0 |
| 11 | 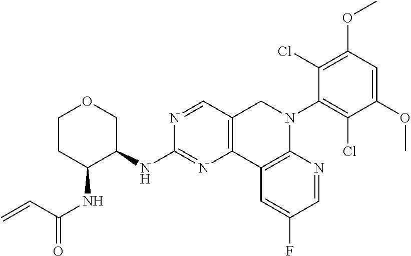 [SMILES: C=CC(=O)N[C@H]1CCOC[C@H]1Nc1ncc2c(n1)-c1cc(F)cnc1N(c1c(Cl)c(OC)cc(OC)c1Cl)C2] | N-((3S,4S)-3-((6-(2,6-dichloro-3,5-dimethoxy- phenyl)-9-fluoro-5,6-dihydropyrimido[5,4-c] [1,8]naphthyridin-2-yl)amino)tetrahydro-2H- pyran-4-yl)acrylamide | 575.7 |
| 12 | 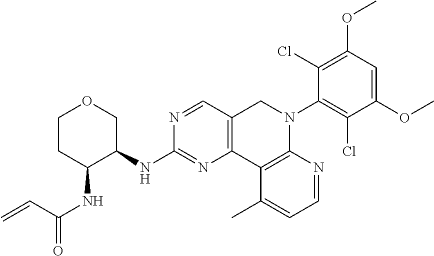 [SMILES: C=CC(=O)N[C@H]1CCOC[C@H]1Nc1ncc2c(n1)-c1c(C)ccnc1N(c1c(Cl)c(OC)cc(OC)c1Cl)C2] | N-((3S,4S)-3-((6-(2,6-dichloro-3,5-dimethoxy- phenyl)-10-methyl-5,6-dihydropyrimido[5,4-c] [1,8]naphthyridin-2-yl)amino)tetrahydro-2H- pyran-4-yl)acrylamide | 571.7 |
| 13 | 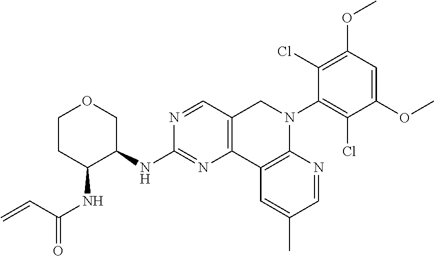 [SMILES: C=CC(=O)N[C@H]1CCOC[C@H]1Nc1ncc2c(n1)-c1cc(C)cnc1N(c1c(Cl)c(OC)cc(OC)c1Cl)C2] | N-((3S,4S)-3-((6-(2,6-dichloro-3,5-dimethoxy- phenyl)-9-methyl-5,6-dihydropyrimido[5,4-c] [1,8]naphthyridin-2-yl)amino)tetrahydro-2H- pyran-4-yl)acrylamide | 571.7 |
| 14 | 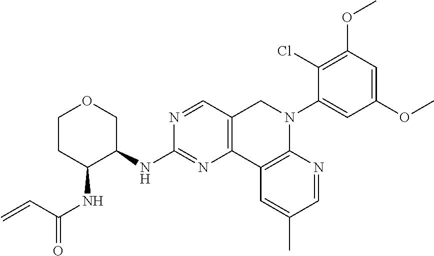 [SMILES: C=CC(=O)N[C@H]1CCOC[C@H]1Nc1ncc2c(n1)-c1cc(C)cnc1N(c1cc(OC)cc(OC)c1Cl)C2] | N-((3S,4S)-3-((6-(2-chloro-3,5-dimethoxy- phenyl)-9-methyl-5,6-dihydropyrimido[5,4-c][1,8] naphthyridin-2-yl)amino)tetrahydro-2H-pyran- 4-yl)acrylamide | 537.7 |
| 15 | 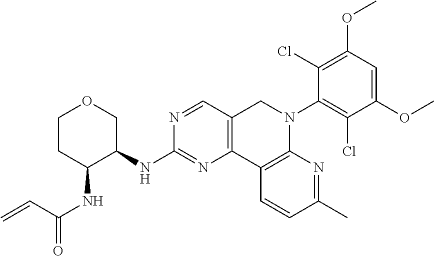 [SMILES: C=CC(=O)N[C@H]1CCOC[C@H]1Nc1ncc2c(n1)-c1ccc(C)nc1N(c1c(Cl)c(OC)cc(OC)c1Cl)C2] | N-((3S,4S)-3-((6-(2,6-dichloro-3,5-dimethoxy- phenyl)-8-methyl-5,6-dihydropyrimido[5,4-c] [1,8]naphthyridin-2-yl)amino)tetrahydro-2H- pyran-4-yl)acrylamide | 571.7 |
| 16 | 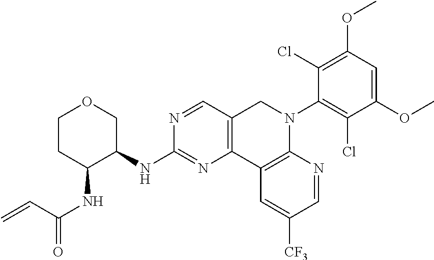 [SMILES: C=CC(=O)N[C@H]1CCOC[C@H]1Nc1ncc2c(n1)-c1cc(C(F)(F)F)cnc1N(c1c(Cl)c(OC)cc(OC)c1Cl)C2] | N-((3S,4S)-3-((6-(2,6-dichloro-3,5-dimethoxy- phenyl)-9-(trifluoromethyl)-5,6-dihydropyrimido [5,4-c][1,8]naphthyridin-2-yl)amino)tetra- hydro-2H-pyran-4-yl)acrylamide | 625.6 |
| 17 | 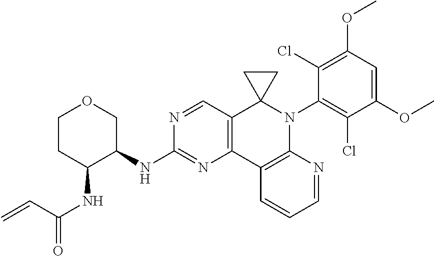 [SMILES: C=CC(=O)N[C@H]1CCOC[C@H]1Nc1ncc2c(n1)-c1cccnc1N(c1c(Cl)c(OC)cc(OC)c1Cl)C21CC1] | N-((2S,4S)-3-((6′-(2,6-dichloro-3,5-dimethoxy- phenyl)-6′H-spiro[cyclopropane-1,5′-pyrimido [5,4-c][1,8]naphthyridin]-2′-yl)amino)tetrahydro- 2H-pyran-4-yl)acrylamide | 583.1 |
| 18 | 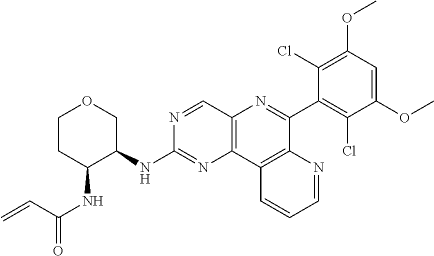 [SMILES: C=CC(=O)N[C@H]1CCOC[C@H]1Nc1ncc2nc(-c3c(Cl)c(OC)cc(OC)c3Cl)c3ncccc3c2n1] | N-((3S,4S)-3-((6-(2,6-dichloro-3,5-dimethoxy- phenyl)pyrimido[4,5-f][1,7]naphthyridin-2-yl) amino)tetrahydro-2H-pyran-4-yl)acrylamide | 555.1 |
| 19 | 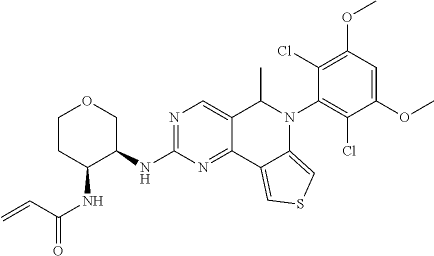 [SMILES: C=CC(=O)N[C@H]1CCOC[C@H]1Nc1ncc2c(n1)-c1cscc1N(c1c(Cl)c(OC)cc(OC)c1Cl)C2C] | N-((3S,4S)-3-((6-(2,6-dichloro-3,5-dimethoxy- phenyl)-5-methyl-5,6-dihydrothieno[3′,4′:5,6] pyrido[4,3-d]pyrimidin-2-yl)amino)tetrahydro- 2H-pyran-4-yl)acrylamide | 576.2 |
| 20 | 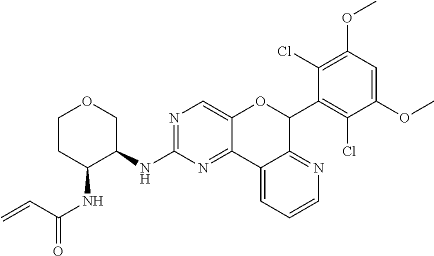 [SMILES: C=CC(=O)N[C@H]1CCOC[C@H]1Nc1ncc2c(n1)-c1cccnc1C(c1c(Cl)c(OC)cc(OC)c1Cl)O2] | N-((3S,4S)-3-((6-(2,6-dichloro-3,5-dimethoxy- phenyl)-6H-pyrido[3′,2′:4,5]pyrano[3,2-d] pyrimidin-2-yl)amino)tetrahydro-2H-pyran-4-yl) acrylamide | 558.1 |
| 21 | 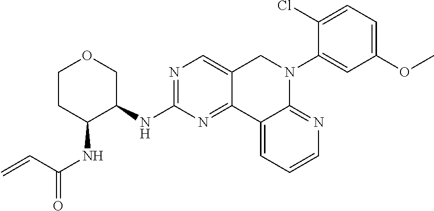 [SMILES: C=CC(=O)N[C@H]1CCOC[C@H]1Nc1ncc2c(n1)-c1cccnc1N(c1cc(OC)ccc1Cl)C2] | N-((3S,4S)-3-((6-(2-chloro-5-methoxyphenyl)- 5,6-dihydropyrimido[5,4-c][1,8]naphthyridin- 2-yl)amino)tetrahydro-2H-pyran-4-yl)acrylamide | 493.1 |
| 22 | 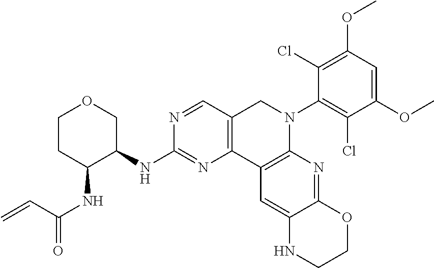 [SMILES: C=CC(=O)N[C@H]1CCOC[C@H]1Nc1ncc2c(n1)-c1cc3c(nc1N(c1c(Cl)c(OC)cc(OC)c1Cl)C2)OCCN3] | N-((3S,4S)-3-((6-(2,6-dichloro-3,5-dimethoxy- phenyl)-6,9,10,11-tetrahydro-5H-[1,4]oxazino [2,3-b]pyrimido[4,5-f][1,8]naphthridin-2-yl) amino)tetrahydro-2H-pyran-4-yl)acrylamide | 614.1 |
| 23 | 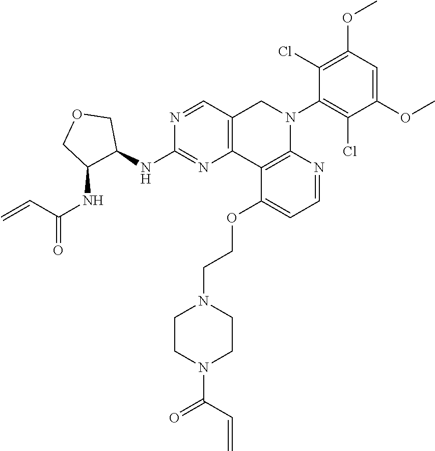 [SMILES: C=CC(=O)N[C@H]1COC[C@H]1Nc1ncc2c(n1)-c1c(OCCN3CCN(C(=O)C=C)CC3)ccnc1N(c1c(Cl)c(OC)cc(OC)c1Cl)C2] | N-((3R,4S)-4-((10-(2-(4-acryloylpiperazin-1- yl)ethoxy)-6-(2,6-dichloro-3,5-dimethoxyphenyl)- 5,6-dihydropyrimido[5,4-c][1,8]naphthyridin- 2-yl)amino)tetrahydrofuran-3-yl)acrylamide | 725.2 |
| 24 | 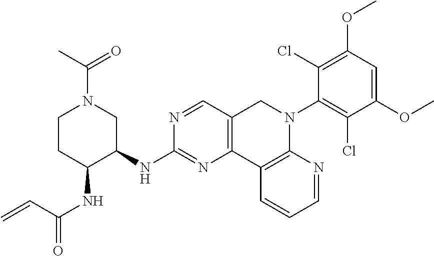 [SMILES: C=CC(=O)N[C@H]1CCN(C(C)=O)C[C@H]1Nc1ncc2c(n1)-c1cccnc1N(c1c(Cl)c(OC)cc(OC)c1Cl)C2] | N-((3R,4S)-1-acetyl-3-((6-(2,6-dichloro-3,5-di- methoxyphenyl)-5,6-dihydropyrimido[5,4-c] [1,8]naphthyridin-2-yl)amino)piperidin-4-yl) acrylamide | 598.1 |
| 25 | 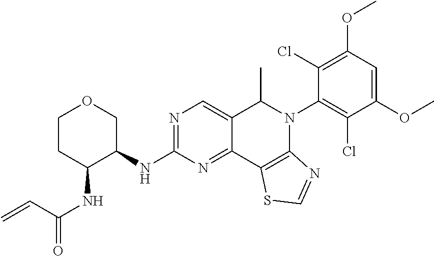 [SMILES: C=CC(=O)N[C@H]1CCOC[C@H]1Nc1ncc2c(n1)-c1scnc1N(c1c(Cl)c(OC)cc(OC)c1Cl)C2C] | N-((3S,4S)-3-((4-(2,6-dichloro-3,5-dimethoxy- phenyl)-5-methyl-4,5-dihydrothiazolo[5′,4′:5,6] pyrido[4,3-d]pyrimidin-8-yl)amino)tetrahydro- 2H-pyran-4-yl)acrylamide | 577.1 |
| 26 | 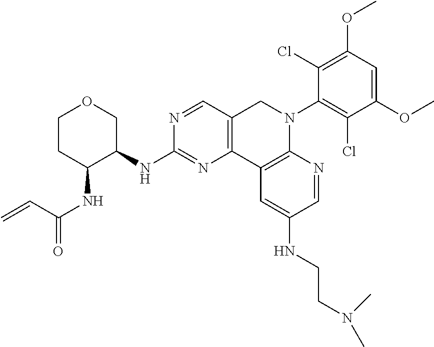 [SMILES: C=CC(=O)N[C@H]1CCOC[C@H]1Nc1ncc2c(n1)-c1cc(NCCN(C)C)cnc1N(c1c(Cl)c(OC)cc(OC)c1Cl)C2] | N-((3S,4S)-3-((6-(2,6-dichloro-3,5-dimethoxy- phenyl)-9-((2-(dimethylamino)ethyl)amino)-5,6- dihydropyrimido[5,4-c][1,8]naphthyridin-2- yl)amino)tetrahydro-2H-pyran-4-yl)acrylamide | 643.2 |
| 27 | 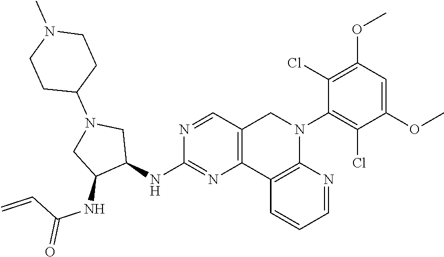 [SMILES: C=CC(=O)N[C@H]1CN(C2CCN(C)CC2)C[C@H]1Nc1ncc2c(n1)-c1cccnc1N(c1c(Cl)c(OC)cc(OC)c1Cl)C2] | N-((3S,4R)-4-((6-(2,6-dichloro-3,5-dimethoxy- phenyl)-5,6-dihydropyrimido[5,4-c][1,8] naphthyridin-2-yl)amino)-1-(1-methylpiperidin-4-yl) pyrrolidin-3-yl)acrylamide | 639.2 |
| 28 | 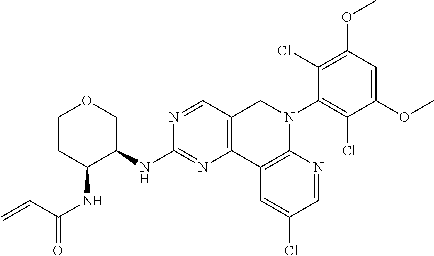 [SMILES: C=CC(=O)N[C@H]1CCOC[C@H]1Nc1ncc2c(n1)-c1cc(Cl)cnc1N(c1c(Cl)c(OC)cc(OC)c1Cl)C2] | N-((3S,4S)-3-((9-chloro-6-(2,6-dichloro-3,5-di- methoxyphenyl)-5,6-dihydropyrimido[5,4-c] [1,8]naphthyridin-2-yl)amino)tetrahydro-2H- pyran-4-yl)acrylamide | 591.6 |
| 29 | 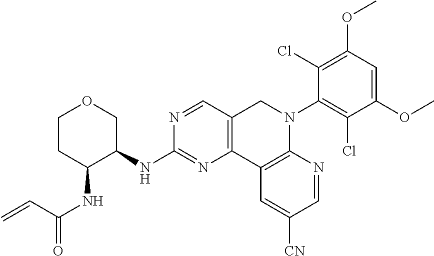 [SMILES: C=CC(=O)N[C@H]1CCOC[C@H]1Nc1ncc2c(n1)-c1cc(C#N)cnc1N(c1c(Cl)c(OC)cc(OC)c1Cl)C2] | N-((3S,4S)-3-((9-cyano-6-(2,6-dichloro-3,5-di- methoxyphenyl)-5,6-dihydropyrimido[5,4-c] [1,8]naphthyridin-2-yl)amino)tetrahydro-2H- pyran-4-yl)acrylamide | 582.8 |
| 30 | 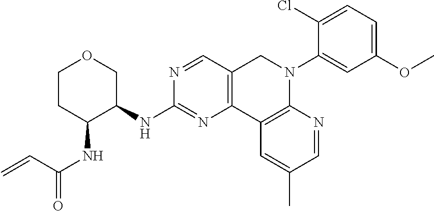 [SMILES: C=CC(=O)N[C@H]1CCOC[C@H]1Nc1ncc2c(n1)-c1cc(C)cnc1N(c1cc(OC)ccc1Cl)C2] | N-((3S,4S)-3-((6-(2-chloro-5-methoxyphenyl)- 9-methyl-5,6-dihydropyrimido[5,4-c][1,8] naphthyridin-2-yl)amino)tetrahydro-2H-pyran-4- yl)acrylamide | 507.2 |
| 31 | 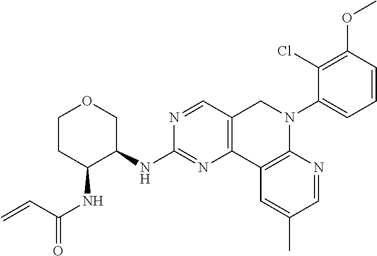 [SMILES: C=CC(=O)N[C@H]1CCOC[C@H]1Nc1ncc2c(n1)-c1cc(C)cnc1N(c1cccc(OC)c1Cl)C2] | N-((3S,4S)-3-((6-(2-chloro-3-methoxyphenyl)- 9-methyl-5,6-dihydropyrimido[5,4-c][1,8] naphthyridin-2-yl)amino)tetrahydro-2H-pyran-4- yl)acrylamide | 507.2 |
| 32 | 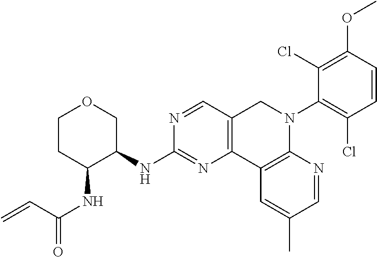 [SMILES: C=CC(=O)N[C@H]1CCOC[C@H]1Nc1ncc2c(n1)-c1cc(C)cnc1N(c1c(Cl)ccc(OC)c1Cl)C2] | N-((3S,4S)-3-((6-(2,6-dichloro-3-methoxy- phenyl)-9-methyl-5,6-dihydropyrimido[5,4-c][1,8] naphthyridin-2-yl)amino)tetrahydro-2H-pyran- 4-yl)acrylamide | 541.1 |
| 33 | 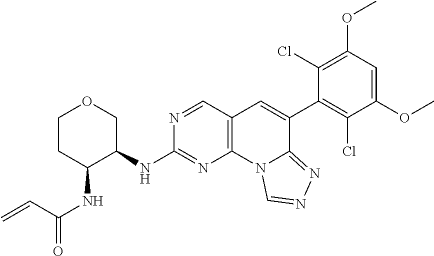 [SMILES: C=CC(=O)N[C@H]1CCOC[C@H]1Nc1ncc2cc(-c3c(Cl)c(OC)cc(OC)c3Cl)c3nncn3c2n1] | N-((3S,4S)-3-((6-(2,6-dichloro-3,5-dimethoxy- phenyl)-[1,2,4]triazolo[4′,3′:1,6]pyrido[2,3-d] pyrimidin-2-yl)amino)tetrahydro-2H-pyran-4- yl)acrylamide | 543.1 |
| 34 | 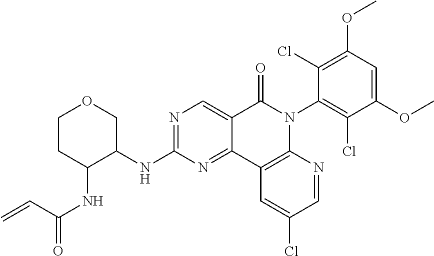 [SMILES: C=CC(=O)NC1CCOCC1Nc1ncc2c(=O)n(-c3c(Cl)c(OC)cc(OC)c3Cl)c3ncc(Cl)cc3c2n1] | N-(3-((9-chloro-6-(2,6-dichloro-3,5-dimethoxy- phenyl)-5-oxo-5,6-dihydropyrimido[5,4-c][1,8] naphthyridin-2-yl)amino)tetrahydro-2H-pyran- 4-yl)acrylamide | 605.9 |

The NMR data of compounds 13, 14, 16, 28 and 32 are as follows:

¹H NMR (DMSO-d6, 500 MHz) δ(ppm) 8.14 (s, 1H), 8.12 (s, 1H), 7.99 (d, J=7.8 Hz, 1H), 7.78 (s, 1H), 6.96 (s, 1H), 6.57 (d, J=7.5 Hz, 1H), 6.19 (br, 1H), 6.05-6.01 (m, 1H), 5.49 (br, 1H), 4.76 (d, J=14.0 Hz, 1H), 4.71 (d, J=14.0 Hz, 1H), 4.43 (br, 1H), 4.27-4.20 (m, 1H), 3.95 (s, 6H), 3.91-3.72 (m, 2H), 3.68-3.57 (m, 1H), 3.56-3.44 (m, 1H), 2.17 (s, 3H), 2.02-1.85 (m, 1H), 1.71-1.54 (m, 1H). (Compound 13);

¹H NMR (CDCl₃, 500 MHZ) δ(ppm) 8.14 (s, 1H), 8.03 (s, 1H), 7.96 (s, 1H), 7.04 (d, J=5.0 Hz, 1H), 6.53 (s, 1H), 6.51 (s, 1H), 6.18 (d, J=17.1 Hz, 1H), 5.90 (dd, J=16.7, 10.9 Hz, 1H), 5.80 (d, J=8.0 Hz, 1H), 5.49 (d, J=10.1 Hz, 1H), 4.81 (s, 2H), 4.45 (s, 1H), 4.30-4.25 (m, 1H), 4.07-4.01 (m, 1H), 3.98 (d, J=11.7 Hz, 1H), 3.90 (s, 3H), 3.80 (s, 3H), 3.73 (d, J=11.8 Hz, 1H), 3.61 (t, J=12.0 Hz, 1H), 2.21 (s, 3H), 2.08 (d, J=12.4 Hz, 1H), 1.87 (tt, J=12.3, 6.3 Hz, 1H). (Compound 14);

¹H NMR (CDCl₃, 500 MHZ) δ(ppm) 8.51 (s, 1H), 8.26 (s, 1H), 8.06 (s, 1H), 6.64 (s, 2H), 6.17 (d, J=17.0 Hz, 1H), 5.91 (t, J=10.9 Hz, 2H), 5.51 (d, J=10.1 Hz, 1H), 4.87 (d, J=15.9 Hz, 2H), 4.49 (br, 1H), 4.30-4.24 (m, 2H), 4.01-3.95 (m, 1H), 3.97 (s, 6H), 3.73 (d, J=11.7 Hz, 1H), 3.60 (t, J=11.7 Hz, 1H), 2.14-1.96 (m, 1H), 1.90-1.80 (m, 1H) (Compound 16);

¹H NMR (CDCl₃, 500 MHZ) δ(ppm) 8.27 (s, 1H), 8.04 (s, 1H), 7.94 (s, 1H), 6.72 (br, 1H), 6.62 (s, 1H), 5.91 (dd, J=16.6 Hz, 10.5 Hz, 1H), 5.11 (d, J=8.5 Hz, 1H), 5.53 (d, J=10.0 Hz, 1H), 4.87 (s, 2H), 4.47 (br, 1H), 4.31-4.24 (m, 1H), 4.01-3.93 (m, 2H), 3.96 (s, 6H), 3.72 (d, J=12.0 Hz, 1H), 3.60 (t, J=12.0 Hz, 1H), 2.07-2.01 (m, 1H), 1.87-1.82 (m, 1H) (Compound 28);

¹H NMR (CDCl₃, 500 MHz) δ(ppm) 9.49 (s, 1H), 8.16 (s, 1H), 8.06 (s, 1H), 7.73 (s, 1H), 7.41 (d, J=9.0 Hz, 1H), 6.95 (d, J=8.9 Hz, 2H), 6.22 (d, J=17.0 Hz, 1H), 6.06 (dd, J=17.0, 10.4 Hz, 1H), 5.61 (d, J=10.3 Hz, 1H), 4.88-4.72 (m, 2H), 4.60-4.45 (m, 2H), 4.31 (d, J=11.5 Hz, 1H), 4.20-4.00 (m, 1H), 3.98 (s, 3H), 3.69 (d, J=12.3 Hz, 1H), 3.58 (t, J=11.3 Hz, 1H), 2.29 (s, 3H), 2.26-1.95 (m, 1H), 1.84-1.77 (m, 1H). (Compound 32).

### Pharmacological Test

### Example A: Kinase Assay

The effect of the compounds of the present invention on the activity of tyrosine kinase FGFR4 was evaluated with in vitro kinase detection experiment. The mobility shift assay was used in the experiment, and a fluorescently labeled polypeptide was used as the substrate, the substrate was transformed into a product under the action of the enzyme in the reaction system, and its charge has also changed accordingly. This method can use the difference between the charge of the substrate and the product to separate them, and then detect them separately.

Experiment Procedure:

(1) Compound Preparation:

DMSO solution of compound (300 μM) was diluted to a 100-fold final concentration of DMSO solution in a 384-well plate, 3-fold dilution, 250 nL of the compound with 100-fold final concentration was transferred to the target plate OptiPlate-384F by a dispenser Echo 550. The final concentration of the compound were 3000 nM, 1000 nM, 333.3 nM, 111.1 nM, 37.04 nM, 12.35 nM, 4.115 nM, 1.372 nM, 0.4572 nM, 0.1524 nM, the compound and the enzyme were incubated for 60 mins,

(2) Kinase Reaction:

Prepared 1× kinase buffer, used the 1× kinase buffer to prepare 2.5 fold final concentration of kinase solution, 10 μL of 2.5 fold final concentration kinase solution was added to the compound well and the positive control well respectively, and 10 μL of 1× kinase buffer was added to the negative control well. After centrifugation, the reaction plate was shaken and mixed and incubated at room temperature for 60 mins, a mixture of 25/15 fold final concentration of adenosine triphosphate (ATP) and kinase substrate 22 was prepared with 1× kinase buffer. 15 μL of 25/15 fold final concentration of ATP and substrate mixed solution was added to start the reaction, 384-well plate was centrifuged, mixed, and then incubated at room temperature for 30 minutes, 30 μL of the stop detection solution was added to stop the kinase reaction, and the conversion rate was readed with Caliper EZ Reader after centrifugation and mixing;

(3) Data Analysis:

$$Calculation formula \text{:} inhibition rate \% = \frac{Conversion {\%}_{max} - Conversion \% \_sample}{Conversion {\%}_{max} - Conversion \% \_min} * 100$$

wherein: Conversion %_sample was the conversion rate of the sample; Conversion %_min was the average value of the negative control well, which represent the conversion rate of the well without enzyme activity, Conversion %_max was the average value of the positive control well, which represent the conversion rate of the well without compound inhibition.

Take the log value of the concentration as the X axis, the percentage inhibition rate as the Y axis, and use GraphPad Prism's log(inhibitor) vs. response-Variable slope (four parameters) of the analysis software to fit the dose-response curve to obtain the IC₅₀ value of each compound on the enzyme. The formula is as follows: Y=Bottom+(Top−Bottom)/(1+10\hat{ }((Log IC₅₀−X)*HillSlope)).

The IC₅₀ data of some Examples and BLU554 are shown in Table 2.

TABLE 2

IC₅₀(nM) of compound

| Compound number | FGFR1 | FGFR4 |
|---|---|---|
| Compound 1 | /^{\textcircled{1}} | 9.4 |
| Compound 2 | 912 | 10 |
| Compound 11 | 1531 | 1.8 |
| Compound 13 | 367 | 1.0 |
| Compound 14 | 1435 | 2.6 |
| Compound 15 | 1001 | 5.2 |
| Compound 16 | 166 | 3.6 |
| Compound 28 | 741 | 1.8 |
| BLU554^{\textcircled{2}} | 1480 | 13 |

Note
^{\textcircled{1}}“/” represents to not tested;
^{\textcircled{2}}BLU554 is the No. 40 compound which disclosed by Blueprint Medicines Corporation in WO2015061572.

The compound of the present invention has a great inhibitory effect on FGFR4 kinase, and such compounds have a much stronger inhibitory effect on FGFR4 than that on FGFR1, which represent a great selectivity.

### Example B: Cell Proliferation Assay

In vitro cell assay was used to measure the effects of the compound of the present invention on the proliferation of human liver cancer cells Hep3B cells. The CELL TITER-GLO (CTG) luminescent was used as detection method in the assay, which can detect the number of living cells by quantitatively determining ATP. Because ATP participates in a variety of enzymatic reactions in organisms, it is an indicator of the metabolism of living cells, and its content directly reflects the number and cell state of cells, during the experiment, add CellTiter-Glo™ reagent to the cell culture medium to measure the luminescence, the luminescence value is directly proportional to the amount of ATP, and ATP is positively related to the number of living cells, so cell viability can be inspected by detecting the ATP content.

Experiment Procedure.

(1) Cell Plating:

Take a bottle of Hep3B cells in logarithmic growth phase, the cells were counted after digestion and resuspension, and then were adjusted the cell density and seeded at 180 μL per well (1500 cells/well) into a 96-well plate, the plate was incubated for 24 hrs in 37° C. 5% CO₂ incubator;

(2) Cell Drug Delivery:

The 600 μM test substance dissolved in DMSO was diluted with DMSO in a 1:3 ratio to a 200-fold final concentration solution, then the cell culture medium was diluted 20 fold (10×), and 20 μL of the compound solution was added to the 96 wells containing cells in the plate, the final concentration of the compound from high to low was 3000 nM, 1000 nM, 333.3 nM, 111.1 nM, 37.04 nM, 12.35 nM, 4.115 nM, 1.372 nM, 0.4572 nM, and the well plate was placed in a 37° C. 5% CO₂ incubator for 96 hrs;

(3) CTG detection:

After 96 hrs of incubation, 60 μL of CellTiter-Glo® Luminescent Cell Viability Assay solution was added to each well, gently shaked for 2 mins, continued incubating for 10 mins at room temperature, and the luminescence value of each well on the multifunctional microplate reader was readed.

(4) Data Analysis:

Calculated the inhibition rate base on the luminous value,

Inhibition rate %=(blank group value−administration group value)/(blank group value−apoptosis group value)*100

The log value of the concentration was used as the X axis, and the percentage inhibition rate was used as the Y axis. Log (inhibitor) vs. response-Variable slope (four parameters) of GraphPad Prism's was used to fit the dose-response curve, and the IC₅₀ of the compound to inhibit cell proliferation was calculated.

The experimental data were shown in Table 3.

TABLE 3

| Compound number | IC₅₀(nM) of the compound on Hep3B cells |
|---|---|
| BLU554 | 62.7 |
| Compound 1 | 6.9 |
| Compound 3 | >3000 |
| Compound 11 | 10.6 |
| Compound 13 | 5.1 |
| Compound 14 | 18.7 |
| Compound 16 | 14.5 |
| Compound 28 | 9.9 |
| Compound 33 | 524.4 |
| Compound 34 | 1027.0 |

The preferred compound of the present invention has a good inhibitory effect on the proliferation of Hep3B cells.

### Example C: Xenograft Tumor Models

BALB/c nu/nu female mice were inoculated subcutaneously in the right anterior scapula of 5×10⁶ human hepatocarcinoma cells Hep3B, and the volume ratio of cell suspension to matrigel was 1:1 (0.2/mL/mouse). The mice were grouped according to tumor size until the average tumor volume was 158 mm³. The treatment group was given a test compound solution prepared with an appropriate solvent, and the solvent control group was given a blank solvent. During the treatment, the tumor volume was measured twice a week, and the tumor weight was measured after the last dose to determine the compound activity. The tumor growth inhibition rate (%, TGI) was calculated by comparing the tumor volume and weight of the treatment group and the solvent control group. Body weight measurement, as a routine determination of toxicity, has the same frequency as the tumor volume measurement. In this model, compound of example 13 showed a good anti-tumor activity. For example, when the doses were 50 mg/kg, 100 mg/kg and 200 mg/kg (BID×14), the inhibitory rate of Example 13 compound on tumor volume growth of Hep3B were 73.02%, 86.26% and 90.26% respectively, the inhibitory rate of tumor weight growth of HepB were 84.76%, 92.27% and 98.15% respectively, which shows that compound of Example 13 showed a dose-dependent effect in inhibiting tumor volume and weight. The compound of example 16 also showed a good tumor activity in this model, when the dose was 50 mg/kg (BID×14), the inhibitory rates of inhibiting tumor volume and weight growth were 78.37% and 83.85%, respectively. In addition, during the entire experiment, the animals given Example 13 and Example 16 compounds did not show obvious weight losses, which indicate that, the two compounds are well tolerated under the conditions of the treatment doses.

Although the present invention has been fully described through its embodiments, it is worth noting that various changes and modifications are obvious to those skilled in the art. Such changes and modifications should be included in the range of the appended claims of the present invention.

1. A compound of Formula (I) or a pharmaceutically acceptable salt, solvate, chelate, non-covalent complex, or prodrug thereof, 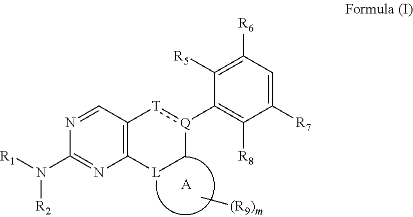 [symbol]
is a single bond or a double bond;
L, Q or T is each independently selected from the group consisting of O, N, C, CH, CH₂ and CR₁₇;
ring A is C₆₋₁₀ aryl, substituted C₆₋₁₀ aryl, C₅₋₁₀ heteroaryl, substituted C₅₋₁₀ heteroaryl, C₅₋₁₀ heterocyclyl, substituted C₅₋₁₀ heterocyclyl; wherein the C₅₋₁₀ heteroaryl or C₅₋₁₀ heterocyclyl optionally containing 1, 2 or 3 heteroatoms independently selected from N, O and S;
R₁ is selected from the group consisting of hydrogen, halogen, C₁₋₈ alkyl, substituted C₁₋₈ alkyl, C₁₋₈ alkoxy, substituted C₁₋₈ alkoxy, C₂₋₈ alkenyl, substituted C₂₋₈ alkenyl, C₂₋₈ alkynyl and substituted C₂₋₈ alkynyl;
R₂ is selected from the group consisting of C₃₋₁₀ cycloalkyl, substituted C₃₋₁₀ cycloalkyl, C₃₋₁₀ heterocyclyl, substituted C₃₋₁₀ heterocyclyl, C₆₋₁₀ aryl, substituted C₆₋₁₀ aryl, C₆₋₁₀ heteroaryl and substituted C₆₋₁₀ heteroaryl, wherein the C₃₋₁₀ heterocyclyl or C₆₋₁₀ heteroaryl optionally containing 1 or 2 heteroatoms selected from N and O;
R₂ is optionally substituted with 1-2 R₁₃ substituents;
R₁₃ is selected from the group consisting of hydroxyl, halogen, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₃₋₁₀ heterocyclyl, substituted C₃₋₁₀ heterocyclyl, C₅₋₁₀ heteroaryl, substituted C₅₋₁₀ heteroaryl, —NR₁₄R₁₅ and —CO—R₁₆, wherein the C₃₋₁₀ heterocyclyl or C₅₋₁₀ heteroaryl optionally containing 1, 2 or 3 heteroatoms selected from N and O;
R₁₃ is optionally substituted with 0-1 R₁₈ substituent;
R₁₈ is selected from the group consisting of hydroxyl, halogen, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₁₋₈ alkoxy, substituted C₁₋₈ alkoxy, C₆₋₁₀ aryl, substituted C_{6- 10} aryl, C₃₋₁₀ cycloalkyl, substituted C₃₋₁₀ cycloalkyl, C₃₋₁₀ heterocyclyl, substituted C₃₋₁₀ heterocyclyl, C₅₋₁₀ heteroaryl and substituted C₅₋₁₀ heteroaryl, wherein the C₃₋₁₀ heterocyclyl or C₅₋₁₀ heteroaryl containing 1, 2 or 3 heteroatoms selected from N and O;
R₅, R₆, R₇ and R₈ is independently selected from the group consisting of hydroxyl, halogen, C₁₋₈ alkoxy, substituted C₁₋₈ alkoxy, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₃₋₈ cycloalkyl, substituted C₃₋₈ cycloalkyl, C₆₋₁₀ aryl, substituted C₆₋₁₀ aryl, C₅₋₁₀ heteroaryl, substituted C₅₋₁₀ heteroaryl, C₃₋₁₀ heterocyclyl and substituted C₃₋₁₀ heterocyclyl;
R₉ is selected from the group consisting of H, halogen, amino, cyano, C₁₋₈ alkyl, substituted C₁₋₈ alkyl, C₁₋₈ alkoxy, substituted C₁₋₈ alkoxy, C₂₋₈ alkenyl, substituted C₂₋₈ alkenyl, C₂₋₈ alkynyl, substituted C₂₋₈ alkynyl, C₆₋₁₀ aryl, substituted C₆₋₁₀ aryl, C₃₋₈ cycloalkyl, substituted C₃₋₈ cycloalkyl, C₃₋₁₀ heterocyclyl, substituted C₃₋₁₀ heterocyclyl, C₅₋₁₀ heteroaryl and substituted C₅₋₁₀ heteroaryl;
R₉ is optionally substituted with 0-1 R₁₀ substituent;
R₁₀ is selected from the group consisting of hydroxyl, halogen, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₁₋₈ alkoxy, substituted C₁₋₈ alkoxy, C₆₋₁₀ aryl, substituted C₆₋₁₀ aryl, C₃₋₈ cycloalkyl, substituted C₃₋₈ cycloalkyl, C₃₋₁₀ heterocyclyl, substituted C₃₋₁₀ heterocyclyl, C₅₋₁₀ heteroaryl, substituted C₅₋₁₀ heteroaryl, —CO—R₁₆ and —(CH₂)ₙNR₁₁R₁₂;
R₁₁ or R₁₂ is optionally selected from the group consisting of H, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₁₋₈ alkoxy, substituted C₁₋₈ alkoxy, C₃₋₈ cycloalkyl, substituted C₃₋₈ cycloalkyl, C₆₋₁₀ aryl, substituted C₆₋₁₀ aryl, C₅₋₁₀ heteroaryl, substituted C₅₋₁₀ heteroaryl, C₃₋₁₀ heterocyclyl and substituted C₃₋₁₀ heterocyclyl;
R₁₄ or R₁₅ is optionally selected from the group consisting of H, C₁₋₈ alkyl, substituted C₁₋₈ alkyl, C₂₋₈ alkenyl, substituted C₂₋₈ alkenyl, —CO—C₂₋₈ alkenyl, substituted —CO—C₂₋₈ alkenyl, C₂₋₈ alkynyl, substituted C₂₋₈ alkynyl, C₃₋₁₀ cycloalkyl, substituted C₃₋₁₀ cycloalkyl, C₆₋₁₀ aryl, substituted C₆₋₁₀ aryl, C₅₋₁₀ heteroaryl, substituted C₅₋₁₀ heteroaryl, C₃₋₁₀ heterocyclyl and substituted C₃₋₁₀ heterocyclyl;
R₁₆ is optionally selected from the group consisting of C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₃₋₁₀ cycloalkyl, substituted C₃₋₁₀ cycloalkyl, C₆₋₁₀ aryl, substituted C₆₋₁₀ aryl, C₅₋₁₀ heteroaryl, substituted C₅₋₁₀ heteroaryl, C₃₋₁₀ heterocyclyl, substituted C₃₋₁₀ heterocyclyl and —NR₁₁R₁₂;
R₁₇ is selected from the group consisting of oxo, C₁₋₈ alkyl, substituted C₁₋₈ alkyl, C₂₋₈ alkenyl, substituted C₂₋₈ alkenyl, C₂₋₈ alkynyl, substituted C₂₋₈ alkynyl, C₃₋₁₀ cycloalkyl, substituted C₃₋₁₀ cycloalkyl, C₆₋₁₀ aryl, substituted C₆₋₁₀ aryl, C₅₋₁₀ heteroaryl, substituted C₅₋₁₀ heteroaryl, C₃₋₁₀ heterocyclyl, substituted C₃₋₁₀ heterocyclyl, or R₁₇ is C₃₋₁₀ cycloalkyl, taken together with the carbon atom to which they are attached form a spiro ring;
M is 0 or 1;
N is 0, 1 or 2;
and provided that:
if ring A is a 5-member heteroaryl containing 2 or 3 N atoms, then R₂ is vinylamide substituted 6-member heterocyclyl comprising oxygen heteroatom, wherein vinylamide substituted 6-member heterocyclyl optionally substituted with 0-1 R₁₃ substituent;
if T is CR₁₇, and R₁₇ is oxo, then ring A is not a 5-member heteroaryl comprising N heteroatom.

2. The compound or a pharmaceutically acceptable salt, solvate, chelate, non-covalent complex, or prodrug thereof of claim 1, wherein L, Q and T is selected from the following groups:
(i) L is C, Q is N, T is CH₂;
(ii) L is C, Q is N, T is C;
(iii) L is C, Q is C, T is N;
(iv) L is C, Q is N, T is CH;
(v) L is N, Q is N, T is CH₂; or
(vi) L is C, Q is CH, T is O.

3. The compound or a pharmaceutically acceptable salt, solvate, chelate, non-covalent complex, or prodrug thereof of claim 1, wherein the compound is shown as formula (II):

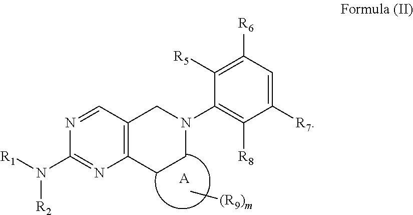

4. The compound or a pharmaceutically acceptable salt, solvate, chelate, non-covalent complex, or prodrug thereof of claim 1, wherein ring A is phenyl, C₅₋₆ heteroaryl or C₁₀ heterocyclyl, wherein the C₅₋₆ heteroaryl optionally containing 1, 2 or 3 heteroatoms selected from N and S, the C₁₀ heterocyclyl is a fused bicyclic which has two N atoms and one O atom in the ring.

5. The compound or a pharmaceutically acceptable salt, solvate, chelate, non-covalent complex, or prodrug thereof of claim 1, wherein ring A is C₆ heteroaryl.

6. The compound or a pharmaceutically acceptable salt, solvate, chelate, non-covalent complex, or prodrug thereof of claim 1, wherein R₉ is selected from the group consisting of H, halogen, cyano, C₁₋₆ alkyl, halogen substituted C₁₋₆ alkyl, —(CH₂)ₙNR₁₁R₁₂ substituted amino, C₁₋₆ alkoxy which substituted with substituted C₆ heterocyclyl, wherein R₁₁ and R₁₂ are each optionally selected from C₁₋₆ alkyl.

7. The compound or a pharmaceutically acceptable salt, solvate, chelate, non-covalent complex, or prodrug thereof of claim 1, wherein

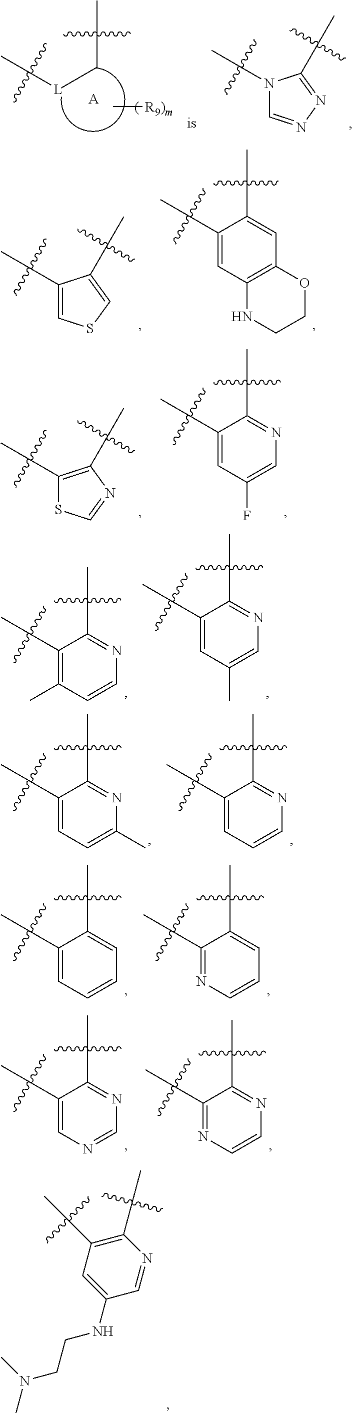

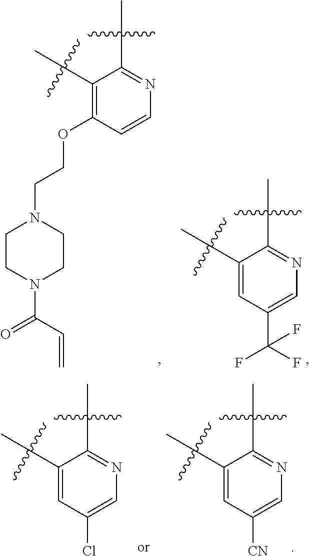 [SMILES: *c1nccc(OCCN2CCN(C(=O)C=C)CC2)c1* ; *c1cc(C(F)(F)F)cnc1* ; *c1cc(Cl)cnc1* ; *c1cc(C#N)cnc1*]

8. The compound or a pharmaceutically acceptable salt, solvate, chelate, non-covalent complex, or prodrug thereof of claim 1, wherein R₁ is hydrogen, R₂ is selected from the group consisting of C₅₋₆ cycloalkyl, substituted C₅₋₆ cycloalkyl, C₅₋₇ heterocyclyl, substituted C₅₋₇ heterocyclyl and phenyl, wherein the C₅₋₇ heterocyclyl optionally containing 1 or 2 heteroatoms selected from N and O.

9. The compound or a pharmaceutically acceptable salt, solvate, chelate, non-covalent complex, or prodrug thereof of claim 1, wherein R₂ is substituted with 1 or 2 R₁₃ substituents, R₁₃ is selected from the group consisting of C₅₋₆ heterocyclyl, substituted C₅₋₆ heterocyclyl, —NR₁₄R₁₅ and —CO—R₁₆, R₁₄ is H, R₁₅ is —CO—C₂₋₄ alkenyl, R₁₆ is C₁₋₃ alkyl or substituted C₁₋₃ alkyl.

10. The compound or a pharmaceutically acceptable salt, solvate, chelate, non-covalent complex, or prodrug thereof of claim 1, wherein R₁₃ is substituted with 0-1 R₁₈ substituent, R₁₈ is selected from the group consisting of C₁₋₆ alkyl, C₅₋₆ heterocyclyl and substituted C₅₋₆ heterocyclyl, wherein the C₅₋₆ heterocyclyl containing 1 or 2 heteroatoms selected from N and O.

11. The compound or a pharmaceutically acceptable salt, solvate, chelate, non-covalent complex, or prodrug thereof of claim 1, wherein R₂ is

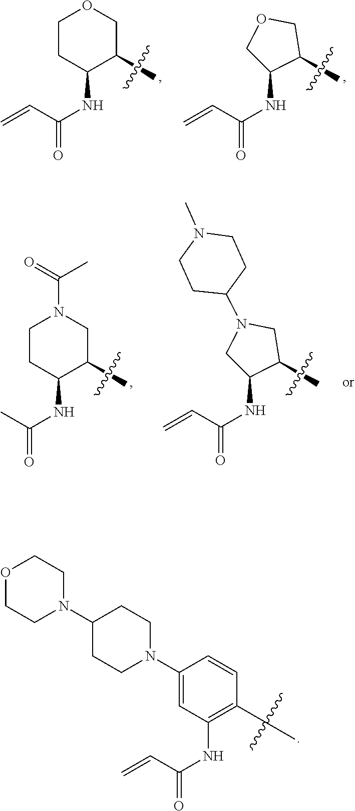

12. The compound or a pharmaceutically acceptable salt, solvate, chelate, non-covalent complex, or prodrug thereof of claim 1, wherein R₅, R₆, R₇ and R₈ are each independently selected from the group consisting of hydrogen, halogen, C₁₋₃ alkoxy and substituted C₁₋₃ alkoxy.

13. The compound or a pharmaceutically acceptable salt, solvate, chelate, non-covalent complex, or prodrug thereof of claim 1, wherein R₅ and R₈ are selected from the following groups:
(i) both R₅ and R₈ are chlorine;
(ii) both R₅ and R₈ are hydrogen;
(iii) R₅ is hydrogen, R₈ is chlorine; or
(iv) R₅ is chlorine, R₈ is hydrogen.

14. The compound or a pharmaceutically acceptable salt, solvate, chelate, non-covalent complex, or prodrug thereof of claim 1, wherein R₆ and R₇ are selected from the following groups:
(i) both R₆ and R₇ are methoxy;
(ii) R₆ is methoxy, R₇ is H; or
(iii) R₆ is H, R₇ is methoxy.

15. The compound or a pharmaceutically acceptable salt, solvate, chelate, non-covalent complex, or prodrug thereof of claim 1, wherein m is 0 or 1.

16. The compound or a pharmaceutically acceptable salt, solvate, chelate, non-covalent complex, or prodrug thereof of claim 1, wherein n is 2.

17. A compound or a pharmaceutically acceptable salt, wherein the compound is selected from:
(1) N-(2-((6-(2,6-dichloro-3,5-dimethoxyphenyl)-5,6-dihydropyrimido[5,4-c][1,8]naphthyridin-2-yl)amino)-5-(4-morpholinopiperidin-1-yl)phenyl)acrylamide;
(2) N-((3S,4S)-3-((6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide;
(3) N-((3S,4S)-3-((6-(2,6-dichloro-3,5-dimethoxyphenyl)-5-oxo-5,6-dihydropyrimido[5,4-c][1,8]naphthyridin-2-yl)amino)tetrahydro-2H-pyran-4-yl)acrylamide;
(4) N-((3S,4S)-3-((6-(2,6-dichloro-3,5-dimethoxyphenyl)-5-methyl-5,6-dihydropyrimido[5,4-c][1,8]naphthyridin-2-yl)amino)tetrahydro-2H-pyran-4-yl)acrylamide;
(5) N-((3S,4S)-3-((6-(2,6-dichloro-3,5-dimethoxyphenyl)-5,6-dihydropyrimido[5,4-e][1,2,4]triazolo[4,3-a]pyrimidin-2-yl)amino)tetrahydro-2H-pyran-4-yl)acrylamide;
(6) N-((3S,4S)-3-((6-(2,6-dichloro-3,5-dimethoxyphenyl)-5,6-dihydropyrimido[5,4-c]quinolin-2-yl)amino)tetrahydro-2H-pyran-4-yl)acrylamide;
(7) N-((3R,4S)-4-((6-(2,6-dichloro-3,5-dimethoxyphenyl)-5,6-dihydropyrimido[5,4-c][1,8]naphthyridin-2-yl)amino)tetrahydrofuran-3-yl)acrylamide;
(8) N-((3S,4S)-3-((6-(2,6-dichloro-3,5-dimethoxyphenyl)-5,6-dihydropyrimido[5,4-c][1,5]naphthyridin-2-yl)amino)tetrahydro-2H-pyran-4-yl)acrylamide;
(9) N-((3S,4S)-3-((6-(2,6-dichloro-3,5-dimethoxyphenyl)-5,6-dihydropyrido[2,3-d:4,5-d′]dipyrimidin-2-yl)amino)tetrahydro-2H-pyran-4-yl)acrylamide;
(10) N-((3S,4S)-3-((6-(2,6-dichloro-3,5-dimethoxyphenyl)-5,6-dihydropyrazino[2′,3′:5,6]pyrido[4,3-d]pyrimidin-2-yl)amino)tetrahydro-2H-pyran-4-yl)acrylamide;
(11) N-((3S,4S)-3-((6-(2,6-dichloro-3,5-dimethoxyphenyl)-9-fluoro-5,6-dihydropyrimido[5,4-c][1,8]naphthyridin-2-yl)amino)tetrahydro-2H-pyran-4-yl)acrylamide;
(12) N-((3S,4S)-3-((6-(2,6-dichloro-3,5-dimethoxyphenyl)-10-methyl-5,6-dihydropyrimido[5,4-c][1,8]naphthyridin-2-yl)amino)tetrahydro-2H-pyran-4-yl)acrylamide;
(13) N-((3S,4S)-3-((6-(2,6-dichloro-3,5-dimethoxyphenyl)-9-methyl-5,6-dihydropyrimido[5,4-c][1,8]naphthyridin-2-yl)amino)tetrahydro-2H-pyran-4-yl)acrylamide;
(14) N-((3S,4S)-3-((6-(2-chloro-3,5-dimethoxyphenyl)-9-methyl-5,6-dihydropyrimido[5,4-c][1,8]naphthyridin-2-yl)amino)tetrahydro-2H-pyran-4-yl)acrylamide;
(15) N-((3S,4S)-3-((6-(2,6-dichloro-3,5-dimethoxyphenyl)-8-methyl-5,6-dihydropyrimido[5,4-c][1,8]naphthyridin-2-yl)amino)tetrahydro-2H-pyran-4-yl)acrylamide;
(16) N-((3S,4S)-3-((6-(2,6-dichloro-3,5-dimethoxyphenyl)-9-(trifluoromethyl)-5,6-dihydropyrimido[5,4-c][1,8]naphthyridin-2-yl)amino)tetrahydro-2H-pyran-4-yl)acrylamide;
(17) N-((3S,4S)-3-((6′-(2,6-dichloro-3,5-dimethoxyphenyl)-6′H-spiro[cyclopropane-1,5′-pyrimido[5,4-c][1,8]naphthyridin]-2′-yl)amino)tetrahydro-2H-pyran-4-yl)acrylamide;
(18) N-((3S,4S)-3-((6-(2,6-dichloro-3,5-dimethoxyphenyl)pyrimido[4,5-f][1,7]naphthyridin-2-yl)amino)tetrahydro-2H-pyran-4-yl)acrylamide;
(19) N-((3S,4S)-3-((6-(2,6-dichloro-3,5-dimethoxyphenyl)-5-methyl-5,6-dihydrothieno[3′,4′:5,6]pyrido[4,3-d]pyrimidin-2-yl)amino)tetrahydro-2H-pyran-4-yl)acrylamide;
(20) N-((3S,4S)-3-((6-(2,6-dichloro-3,5-dimethoxyphenyl)-6H-pyrido[3′,2′:4,5]pyrano[3,2-d]pyrimidin-2-yl)amino)tetrahydro-2H-pyran-4-yl)acrylamide;
(21) N-((3S,4S)-3-((6-(2-chloro-5-methoxyphenyl)-5,6-dihydropyrimido[5,4-c][1,8]naphthyridin-2-yl)amino)tetrahydro-2H-pyran-4-yl)acrylamide;
(22) N-((3S,4S)-3-((6-(2,6-dichloro-3,5-dimethoxyphenyl)-6,9,10,11-tetrahydro-5H-[1,4]oxazino[2,3-b]pyrimido[4,5-f][1,8]naphthyridin-2-yl)amino)tetrahydro-2H-pyran-4-yl)acrylamide;
(23) N-((3R,4S)-4-((10-(2-(4-acryloylpiperazin-1-yl)ethoxy)-6-(2,6-dichloro-3,5-dimethoxyphenyl)-5,6-dihydropyrimido[5,4-c][1,8]naphthyridin-2-yl)amino)tetrahydrofuran-3-yl)acrylamide;
(24) N-((3R,4S)-1-acetyl-3-((6-(2,6-dichloro-3,5-dimethoxyphenyl)-5,6-dihydropyrimido[5,4-c][1,8]naphthyridin-2-yl)amino)piperidin-4-yl)acrylamide;
(25) N-((3S,4S)-3-((4-(2,6-dichloro-3,5-dimethoxyphenyl)-5-methyl-4,5-dihydrothiazolo[5′,4′:5,6]pyrido[4,3-d]pyrimidin-8-yl)amino)tetrahydro-2H-pyran-4-yl)acrylamide;
(26) N-((3S,4S)-3-((6-(2,6-dichloro-3,5-dimethoxyphenyl)-9-((2-(dimethylamino)ethyl)amino)-5,6-dihydropyrimido[5,4-c][1,8]naphthyridin-2-yl)amino)tetrahydro-2H-pyran-4-yl)acrylamide;
(27) N-((3S,4R)-4-((6-(2,6-dichloro-3,5-dimethoxyphenyl)-5,6-dihydropyrimido[5,4-c][1,8]naphthyridin-2-yl)amino)-1-(1-methylpiperidin-4-yl)pyrrolidin-3-yl)acrylamide;
(28) N-((3S,4S)-3-((9-chloro-6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide;
(29) N-((3S,4S)-3-((9-cyano-6-(2,6-dichloro-3,5-dimethoxyphenyl)-5,6-dihydropyrimido[5,4-c][1,8]naphthyridin-2-yl)amino)tetrahydro-2H-pyran-4-yl)acrylamide;
(30) N-((3S,4S)-3-((6-(2-chloro-5-methoxyphenyl)-9-methyl-5,6-dihydropyrimido[5,4-c][1,8]naphthyridin-2-yl)amino)tetrahydro-2H-pyran-4-yl)acrylamide;
(31) N-((3S,4S)-3-((6-(2-chloro-3-methoxyphenyl)-9-methyl-5,6-dihydropyrimido[5,4-c][1,8]naphthyridin-2-yl)amino)tetrahydro-2H-pyran-4-yl)acrylamide;
(32) N-((3S,4S)-3-((6-(2,6-dichloro-3-methoxyphenyl)-9-methyl-5,6-dihydropyrimido[5,4-c][1,8]naphthyridin-2-yl)amino)tetrahydro-2H-pyran-4-yl)acrylamide;
(33) N-((3S,4S)-3-((6-(2,6-dichloro-3,5-dimethoxyphenyl)-[1,2,4]triazolo[4′,3′:1,6]pyrido[2,3-d]pyrimidin-2-yl)amino)tetrahydro-2H-pyran-4-yl)acrylamide; or
(34) N-(3-((9-chloro-6-(2,6-dichloro-3,5-dimethoxyphenyl)-5-oxo-5,6-dihydropyrimido[5,4-c][1,8]naphthyridin-2-yl)amino)tetrahydro-2H-pyran-4-yl)acrylamide.

18. A pharmaceutical composition comprising a therapeutically effect amount of compound or a pharmaceutically acceptable salt, solvate, chelate, non-covalent complex, or prodrug thereof according to claim 1 and at least one pharmaceutically acceptable excipient.

19. A pharmaceutical composition of claim 18, wherein a mass ratio of the said compound and the pharmaceutically acceptable excipient is 0.0001:1-10.

20. (canceled)

21. (canceled)

22. (canceled)

23. (canceled)

24. (canceled)

25. (canceled)

26. A method of treating or preventing disease mediated FGFR4, comprising administering a therapeutically effective amount of the compound or a pharmaceutically acceptable salt, solvate, chelate, non-covalent complex, or prodrug thereof according to claim 1 or a pharmaceutical composition comprising a therapeutically effect amount of compound or a pharmaceutically acceptable salt, solvate, chelate, non-covalent complex, or prodrug thereof according to claim 1 and at least one pharmaceutically acceptable excipient to a subject.

27. The method of claim 26, wherein the disease mediated by FGFR4 is cancer.

28. The method of claim 27, wherein the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, hepatocellular carcinoma, head and neck tumors, hepatobiliary cell carcinoma, myelodysplastic syndrome, malignant glioma, prostate cancer, thyroid cancer, Schwann cell tumor, lung squamous cell carcinoma, lichenoid keratosis, synovial sarcoma, skin cancer, pancreatic cancer, testicular cancer or liposarcoma.

29. A method of treating cancer comprising administering a therapeutical effective amount of a compound or a pharmaceutically acceptable salt, solvate, chelate, non-covalent complex, or prodrug thereof of claim 1 or a pharmaceutical composition comprising a therapeutically effect amount of compound or a pharmaceutically acceptable salt, solvate, chelate, non-covalent complex, or prodrug thereof of claim 1 and at least one pharmaceutically acceptable excipient to a subject, wherein the cancer is breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, hepatocellular carcinoma, head and neck tumors, hepatobiliary cell carcinoma, myelodysplastic syndrome, malignant glioma, prostate cancer, thyroid cancer, Schwann cell tumor, lung squamous cell carcinoma, lichenoid keratosis, synovial sarcoma, skin cancer, pancreatic cancer, testicular cancer or liposarcoma.

30. The method of claim 26, wherein the subject is human.
